(19) United States
(12) Patent Application Publication
(10) Pub. No.: US 2003/0179888 A1
(43) Pub. Date: Sep. 25, 2003

(54) Voice activity detection (VAD) devices and methods for use with noise suppression systems

(76) Inventors: Gregory C. Burnett, Livermore, CA (US); Nicolas J. Petit, San Francisco, CA (US); Alexander M. Asseily, San Francisco, CA (US); Andrew E. Einaudi, San Francisco, CA (US)

Correspondence Address: Shemwell Gregory & Courtney LLP, Suite 201, 4880 Stevens Creek Boulevard, San Jose, CA, 95129, US

(21) Appl. No.: 10/383,162

(22) Filed: Mar. 5, 2003

Related U.S. Application Data
(60) Provisional application No. 60/362,162, filed on Mar. 5, 2002.
(60) Provisional application No. 60/362,170, filed on Mar. 5, 2002.
(60) Provisional application No. 60/361,981, filed on Mar. 5, 2002.
(60) Provisional application No. 60/362,161, filed on Mar. 5, 2002.
(60) Provisional application No. 60/362,103, filed on Mar. 5, 2002.
(60) Provisional application No. 60/368,343, filed on Mar. 27, 2002.

(51) Int. Cl. A61F 11/06; G10K 11/16; H03B 29/00

(52) U.S. Cl. 381/71.8; 381/71.1

(57) ABSTRACT

Voice Activity Detection (VAD) devices, systems and methods are described for use with signal processing systems to denoise acoustic signals. Components of a signal processing system and/or VAD system receive acoustic signals and voice activity signals. Control signals are automatically generated from data of the voice activity signals. Components of the signal processing system and/or VAD system use the control signals to automatically select a denoising method appropriate to data of frequency subbands of the acoustic signals. The selected denoising method is applied to the acoustic signals to generate denoised acoustic signals.

### RELATED APPLICATIONS

This application claims priority from the following U.S. patent applications: application Ser. No. 60/362,162, entitled PATHFINDER-BASED VOICE ACTIVITY DETECTION (PVAD) USED WITH PATHFINDER NOISE SUPPRESSION, filed Mar. 5, 2002; application Ser. No. 60/362,170, entitled ACCELEROMETER-BASED VOICE ACTIVITY DETECTION (PVAD) WITH PATHFINDER NOISE SUPPRESSION, filed Mar. 5, 2002; application Ser. No. 60/361,981, entitled ARRAY-BASED VOICE ACTIVITY DETECTION (AVAD) AND PATHFINDER NOISE SUPPRESSION, filed Mar. 5, 2002; application Ser. No. 60/362,161, entitled PATHFINDER NOISE SUPPRESSION USING AN EXTERNAL VOICE ACTIVITY DETECTION (VAD) DEVICE, filed Mar. 5, 2002; application Ser. No. 60/362,103, entitled ACCELEROMETER-BASED VOICE ACTIVITY DETECTION, filed Mar. 5, 2002; and application Ser. No. 60/368,343, entitled TWO-MICROPHONE FREQUENCY-BASED VOICE ACTIVITY DETECTION, filed Mar. 27, 2002, all of which are currently pending.

Further, this application relates to the following U.S. patent applications: application Ser. No. 09/905,361, entitled METHOD AND APPARATUS FOR REMOVING NOISE FROM ELECTRONIC SIGNALS, filed Jul. 12, 2001; application Ser. No. 10/159,770, entitled DETECTING VOICED AND UNVOICED SPEECH USING BOTH ACOUSTIC AND NONACOUSTIC SENSORS, filed May 30, 2002; and application Ser. No. 10/301,237, entitled METHOD AND APPARATUS FOR REMOVING NOISE FROM ELECTRONIC SIGNALS, filed Nov. 21, 2002.

### TECHNICAL FIELD

The disclosed embodiments relate to systems and methods for detecting and processing a desired signal in the presence of acoustic noise.

### BACKGROUND

Many noise suppression algorithms and techniques have been developed over the years. Most of the noise suppression systems in use today for speech communication systems are based on a single-microphone spectral subtraction technique first develop in the 1970's and described, for example, by S. F. Boll in “Suppression of Acoustic Noise in Speech using Spectral Subtraction,” IEEE Trans. on ASSP, pp. 113-120, 1979. These techniques have been refined over the years, but the basic principles of operation have remained the same. See, for example, U.S. Pat. No. 5,687,243 of McLaughlin, et al., and U.S. Pat. No. 4,811,404 of Vilmur, et al. Generally, these techniques make use of a single-microphone Voice Activity Detector (VAD) to determine the background noise characteristics, where “voice” is generally understood to include human voiced speech, unvoiced speech, or a combination of voiced and unvoiced speech.

The VAD has also been used in digital cellular systems. As an example of such a use, see U.S. Pat. No. 6,453,291 of Ashley, where a VAD configuration appropriate to the front-end of a digital cellular system is described. Further, some Code Division Multiple Access (CDMA) systems utilize a VAD to minimize the effective radio spectrum used, thereby allowing for more system capacity. Also, Global System for Mobile Communication (GSM) systems can include a VAD to reduce co-channel interference and to reduce battery consumption on the client or subscriber device.

These typical single-microphone VAD systems are significantly limited in capability as a result of the analysis of acoustic information received by the single microphone, wherein the analysis is performed using typical signal processing techniques. In particular, limitations in performance of these single-microphone VAD systems are noted when processing signals having a low signal-to-noise ratio (SNR), and in settings where the background noise varies quickly. Thus, similar limitations are found in noise suppression systems using these single-microphone VADs.

### BRIEF DESCRIPTION OF THE FIGURES

FIG. 1 is a block diagram of a signal processing system including the Pathfinder noise suppression system and a VAD system, under an embodiment.

FIG. 1A is a block diagram of a VAD system including hardware for use in receiving and processing signals relating to VAD, under an embodiment.

FIG. 1B is a block diagram of a VAD system using hardware of the associated noise suppression system for use in receiving VAD information, under an alternative embodiment.

FIG. 2 is a block diagram of a signal processing system that incorporates a classical adaptive noise cancellation system, as known in the art.

FIG. 3 is a flow diagram of a method for determining voiced and unvoiced speech using an accelerometer-based VAD, under an embodiment.

FIG. 4 shows plots including a noisy audio signal (live recording) along with a corresponding accelerometer-based VAD signal, the corresponding accelerometer output signal, and the denoised audio signal following processing by the Pathfinder system using the VAD signal, under an embodiment.

FIG. 5 shows plots including a noisy audio signal (live recording) along with a corresponding SSM-based VAD signal, the corresponding SSM output signal, and the denoised audio signal following processing by the Pathfinder system using the VAD signal, under an embodiment.

FIG. 6 shows plots including a noisy audio signal (live recording) along with a corresponding GEMS-based VAD signal, the corresponding GEMS output signal, and the denoised audio signal following processing by the Pathfinder system using the VAD signal, under an embodiment.

FIG. 7 shows plots including recorded spoken acoustic data with digitally added noise along with a corresponding EGG-based VAD signal, and the corresponding highpass filtered EGG output signal, under an embodiment.

FIG. 8 is a flow diagram 80 of a method for determining voiced speech using a video-based VAD, under an embodiment.

FIG. 9 shows plots including a noisy audio signal (live recording) along with a corresponding single (gradient) microphone-based VAD signal, the corresponding gradient microphone output signal, and the denoised audio signal following processing by the Pathfinder system using the VAD signal, under an embodiment.

FIG. 10 shows a single cardioid unidirectional microphone of the microphone array, along with the associated spatial response curve, under an embodiment.

FIG. 11 shows a microphone array of a PVAD system, under an embodiment.

FIG. 12 is a flow diagram of a method for determining voiced and unvoiced speech using H₁(z) gain values, under an alternative embodiment of the PVAD.

FIG. 13 shows plots including a noisy audio signal (live recording) along with a corresponding microphone-based PVAD signal, the corresponding PVAD gain versus time signal, and the denoised audio signal following processing by the Pathfinder system using the PVAD signal, under an embodiment.

FIG. 14 is a flow diagram of a method for determining voiced and unvoiced speech using a stereo VAD, under an embodiment.

FIG. 15 shows plots including a noisy audio signal (live recording) along with a corresponding SVAD signal, and the denoised audio signal following processing by the Pathfinder system using the SVAD signal, under an embodiment.

FIG. 16 is a flow diagram of a method for determining voiced and unvoiced speech using an AVAD, under an embodiment.

FIG. 17 shows plots including audio signals and from each microphone of an AVAD system along with the corresponding combined energy signal, under an embodiment.

FIG. 18 is a block diagram of a signal processing system including the Pathfinder noise suppression system and a single-microphone (conventional) VAD system, under an embodiment.

FIG. 19 is a flow diagram of a method for generating voicing information using a single-microphone VAD, under an embodiment.

FIG. 20 is a flow diagram of a method for determining voiced and unvoiced speech using an airflow-based VAD, under an embodiment.

FIG. 21 shows plots including a noisy audio signal along with a corresponding manually activated/calculated VAD signal, and the denoised audio signal following processing by the Pathfinder system using the manual VAD signal, under an embodiment.

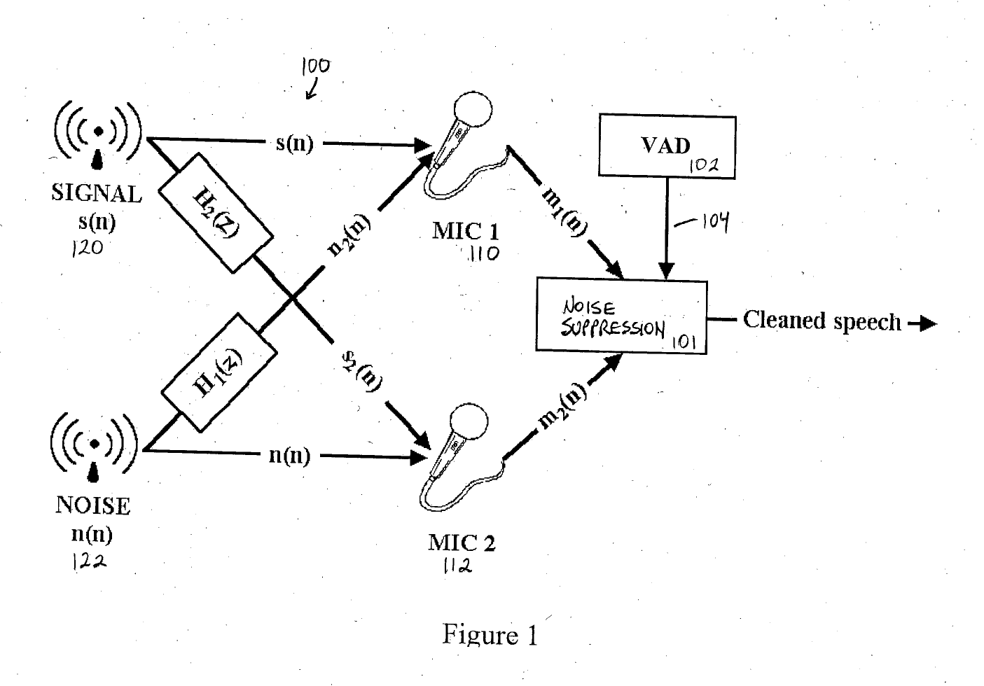

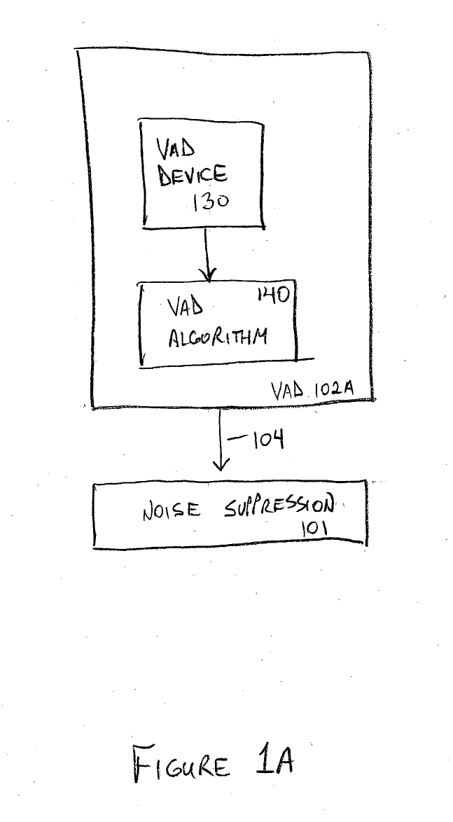

In the drawings, the same reference numbers identify identical or substantially similar elements or acts. To easily identify the discussion of any particular element or act, the most significant digit or digits in a reference number refer to the Figure number in which that element is first introduced (e.g., element 104 is first introduced and discussed with respect to FIG. 1).

### DETAILED DESCRIPTION

Numerous Voice Activity Detection (VAD) devices and methods are described below for use with adaptive noise suppression systems. Further, results are presented below from experiments using the VAD devices and methods described herein as a component of a noise suppression system, in particular the Pathfinder Noise Suppression System available from Aliph, San Francisco, Calif. (http://www.aliph.com), but the embodiments are not so limited. In the description below, when the Pathfinder noise suppression system is referred to, it should be kept in mind that noise suppression systems that estimate the noise waveform and subtract it from a signal and that use or are capable of using VAD information for reliable operation are included in that reference. Pathfinder is simply a convenient referenced implementation for a system that operates on signals comprising desired speech signals along with noise.

When using the VAD devices and methods described herein with a noise suppression system, the VAD signal is processed independently of the noise suppression system, so that the receipt and processing of VAD information is independent from the processing associated with the noise suppression, but the embodiments are not so limited. This independence is attained physically (i.e., different hardware for use in receiving and processing signals relating to the VAD and the noise suppression), through processing (i.e., using the same hardware to receive signals into the noise suppression system while using independent techniques (software, algorithms, routines) to process the received signals), and through a combination of different hardware and different software.

In the following description, “acoustic” is generally defined as acoustic waves propagating in air. Propagation of acoustic waves in media other than air will be noted as such. References to “speech” or “voice” generally refer to human speech including voiced speech, unvoiced speech, and/or a combination of voiced and unvoiced speech. Unvoiced speech or voiced speech is distinguished where necessary. The term “noise suppression” generally describes any method by which noise is reduced or eliminated in an electronic signal.

Moreover, the term “VAD” is generally defined as a vector or array signal, data, or information that in some manner represents the occurrence of speech in the digital or analog domain. A common representation of VAD information is a one-bit digital signal sampled at the same rate as the corresponding acoustic signals, with a zero value representing that no speech has occurred during the corresponding time sample, and a unity value indicating that speech has occurred during the corresponding time sample. While the embodiments described herein are generally described in the digital domain, the descriptions are also valid for the analog domain.

The VAD devices/methods described herein generally include vibration and movement sensors, acoustic sensors, and manual VAD devices, but are not so limited. In one embodiment, an accelerometer is placed on the skin for use in detecting skin surface vibrations that correlate with human speech. These recorded vibrations are then used to calculate a VAD signal for use with or by an adaptive noise suppression algorithm in suppressing environmental acoustic noise from a simultaneously (within a few milliseconds) recorded acoustic signal that includes both speech and noise.

Another embodiment of the VAD devices/methods described herein includes an acoustic microphone modified with a membrane so that the microphone no longer efficiently detects acoustic vibrations in air. The membrane, though, allows the microphone to detect acoustic vibrations in objects with which it is in physical contact (allowing a good mechanical impedance match), such as human skin. That is, the acoustic microphone is modified in some way such that it no longer detects acoustic vibrations in air (where it no longer has a good physical impedance match), but only in objects with which the microphone is in contact. This configures the microphone, like the accelerometer, to detect vibrations of human skin associated with the speech production of that human while not efficiently detecting acoustic environmental noise in the air. The detected vibrations are processed to form a VAD signal for use in a noise suppression system, as detailed below.

Yet another embodiment of the VAD described herein uses an electromagnetic vibration sensor, such as a radiofrequency vibrometer (RF) or laser vibrometer, which detect skin vibrations. Further, the RF vibrometer detects the movement of tissue within the body, such as the inner surface of the cheek or the tracheal wall. Both the exterior skin and internal tissue vibrations associated with speech production can be used to form a VAD signal for use in a noise suppression system as detailed below.

Further embodiments of the VAD devices/methods described herein include an electroglottograph (EGG) to directly detect vocal fold movement. The EGG is an alternating current—(AC) based method of measuring vocal fold contact area. When the EGG indicates sufficient vocal fold contact the assumption that follows is that voiced speech is occurring, and a corresponding VAD signal representative of voiced speech is generated for use in a noise suppression system as detailed below. Similarly, an additional VAD embodiment uses a video system to detect movement of a person's vocal articulators, an indication that speech is being produced.

Another set of VAD devices/methods described below use signals received at one or more acoustic microphones along with corresponding signal processing techniques to produce VAD signals accurately and reliably under most environmental noise conditions. These embodiments include simple arrays and co-located (or nearly so) combinations of omnidirectional and unidirectional acoustic microphones. The simplest configuration in this set of VAD embodiments includes the use of a single microphone, located very close to the mouth of the user in order to record signals at a relatively high SNR. This microphone can be a gradient or “close-talk” microphone, for example. Other configurations include the use of combinations of unidirectional and omnidirectional microphones in various orientations and configurations. The signals received at these microphones, along with the associated signal processing, are used to calculate a VAD signal for use with a noise suppression system, as described below. Also described below is a VAD system that is activated manually, as in a walkie-talkie, or by an observer to the system.

As referenced above, the VAD devices and methods described herein are for use with noise suppression systems like, for example, the Pathfinder Noise Suppression System (referred to herein as the “Pathfinder system”) available from Aliph of San Francisco, Calif. While the descriptions of the VAD devices herein are provided in the context of the Pathfinder Noise Suppression System, those skilled in the art will recognize that the VAD devices and methods can be used with a variety of noise suppression systems and methods known in the art.

The Pathfinder system is a digital signal processing—(DSP) based acoustic noise suppression and echo-cancellation system. The Pathfinder system, which can couple to the front-end of speech processing systems, uses VAD information and received acoustic information to reduce or eliminate noise in desired acoustic signals by estimating the noise waveform and subtracting it from a signal including both speech and noise. The Pathfinder system is described further below and in the Related Applications.

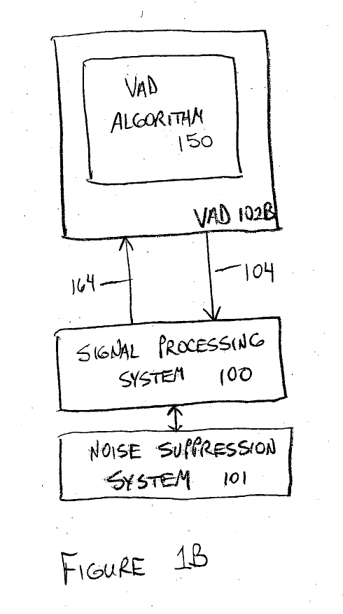

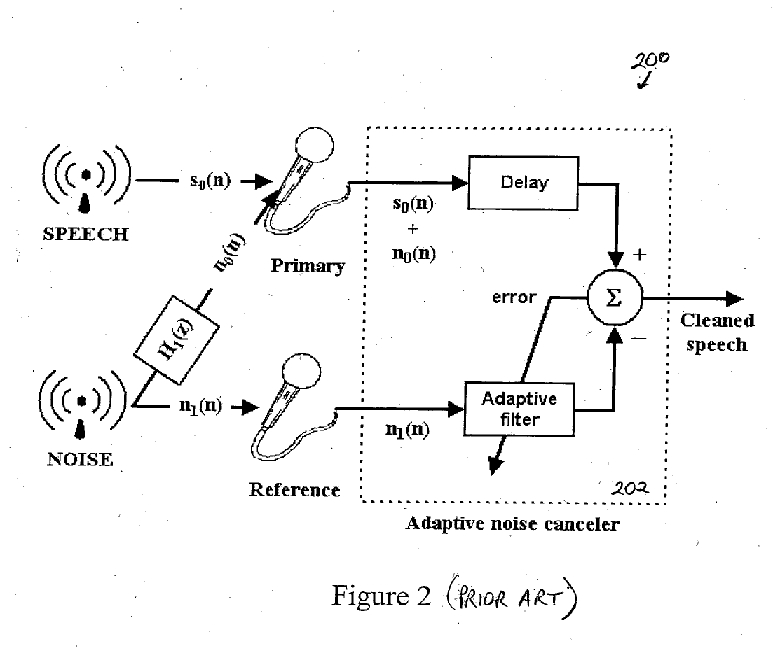

FIG. 1 is a block diagram of a signal processing system 100 including the Pathfinder noise suppression system 101 and a VAD system 102, under an embodiment. The signal processing system 100 includes two microphones MIC 1 110 and MIC 2 112 that receive signals or information from at least one speech signal source 120 and at least one noise source 122. The path s(n) from the speech signal source 120 to MIC 1 and the path n(n) from the noise source 122 to MIC 2 are considered to be unity. Further, H₁(z) represents the path from the noise source 122 to MIC 1, and H₂(z) represents the path from the speech signal source 120 to MIC 2. In contrast to the signal processing system 100 including the Pathfinder system 101, FIG. 2 is a block diagram of a signal processing system 200 that incorporates a classical adaptive noise cancellation system 202 as known in the art.

Components of the signal processing system 100, for example the noise suppression system 101, couple to the microphones MIC 1 and MIC 2 via wireless couplings, wired couplings, and/or a combination of wireless and wired couplings. Likewise, the VAD system 102 couples to components of the signal processing system 100, like the noise suppression system 101, via wireless couplings, wired couplings, and/or a combination of wireless and wired couplings. As an example, the VAD devices and microphones described below as components of the VAD system 102 can comply with the Bluetooth wireless specification for wireless communication with other components of the signal processing system, but are not so limited.

Referring to FIG. 1, the VAD signal 104 from the VAD system 102, derived in a manner described herein, controls noise removal from the received signals without respect to noise type, amplitude, and/or orientation. When the VAD signal 104 indicates an absence of voicing, the Pathfinder system 101 uses MIC 1 and MIC 2 signals to calculate the coefficients for a model of transfer function H₁(z) over pre-specified subbands of the received signals. When the VAD signal 104 indicates the presence of voicing, the Pathfinder system 101 stops updating H₁(z) and starts calculating the coefficients for transfer function H₂(z) over pre-specified subbands of the received signals. Updates of H₁ coefficients can continue in a subband during speech production if the SNR in the subband is low (note that H₁(z) and H₂(z) are sometimes referred to herein as H₁ and H₂, respectively, for convenience). The Pathfinder system 101 of an embodiment uses the Least Mean Squares (LMS) technique to calculate H₁ and H₂, as described further by B. Widrow and S. Stearns in “Adaptive Signal Processing”, Prentice-Hall Publishing, ISBN 0-13-004029-0, but is not so limited. The transfer function can be calculated in the time domain, frequency domain, or a combination of both the time/frequency domains. The Pathfinder system subsequently removes noise from the received acoustic signals of interest using combinations of the transfer functions H₁(z) and H₂(z), thereby generating at least one denoised acoustic stream.

The Pathfinder system can be implemented in a variety of ways, but common to all of the embodiments is reliance on an accurate and reliable VAD device and/or method. The VAD device/method should be accurate because the Pathfinder system updates its filter coefficients when there is no speech or when the SNR during speech is low. If sufficient speech energy is present during coefficient update, subsequent speech with similar spectral characteristics can be suppressed, an undesirable occurrence. The VAD device/method should be robust to support high accuracy under a variety of environmental conditions. Obviously, there are likely to be some conditions under which no VAD device/method will operate satisfactorily, but under normal circumstances the VAD device/method should work to provide maximum noise suppression with few adverse affects on the speech signal of interest.

When using VAD devices/methods with a noise suppression system, the VAD signal is processed independently of the noise suppression system, so that the receipt and processing of VAD information is independent from the processing associated with the noise suppression, but the embodiments are not so limited. This independence is attained physically (i.e., different hardware for use in receiving and processing signals relating to the VAD and the noise suppression), through processing (i.e., using the same hardware to receive signals into the noise suppression system while using independent techniques (software, algorithms, routines) to process the received signals), and through a combination of different hardware and different software, as described below.

FIG. 1A is a block diagram of a VAD system 102A including hardware for use in receiving and processing signals relating to VAD, under an embodiment. The VAD system 102A includes a VAD device 130 coupled to provide data to a corresponding VAD algorithm 140. Note that noise suppression systems of alternative embodiments can integrate some or all functions of the VAD algorithm with the noise suppression processing in any manner obvious to those skilled in the art.

FIG. 1B is a block diagram of a VAD system 102B using hardware of the associated noise suppression system 101 for use in receiving VAD information 164, under an embodiment. The VAD system 102B includes a VAD algorithm 150 that receives data 164 from MIC 1 and MIC 2, or other components, of the corresponding signal processing system 100. Alternative embodiments of the noise suppression system can integrate some or all functions of the VAD algorithm with the noise suppression processing in any manner obvious to those skilled in the art.

Vibration/Movement-Based VAD Devices/Methods

The vibration/movement-based VAD devices include the physical hardware devices for use in receiving and processing signals relating to the VAD and the noise suppression. As a speaker or user produces speech, the resulting vibrations propagate through the tissue of the speaker and, therefore can be detected on and beneath the skin using various methods. These vibrations are an excellent source of VAD information, as they are strongly associated with both voiced and unvoiced speech (although the unvoiced speech vibrations are much weaker and more difficult to detect) and generally are only slightly affected by environmental acoustic noise (some devices/methods, for example the electromagnetic vibrometers described below, are not affected by environmental acoustic noise). These tissue vibrations or movements are detected using a number of VAD devices including, for example, accelerometer-based devices, skin surface microphone (SSM) devices, electromagnetic (EM) vibrometer devices including both radio frequency (RF) vibrometers and laser vibrometers, direct glottal motion measurement devices, and video detection devices.

Accelerometer-Based VAD Devices/Methods

Accelerometers can detect skin vibrations associated with speech. As such, and with reference to FIG. 1 and FIG. 1A, a VAD system 102A of an embodiment includes an accelerometer-based device 130 providing data of the skin vibrations to an associated algorithm 140. The algorithm of an embodiment uses energy calculation techniques along with a threshold comparison, as described below, but is not so limited. Note that more complex energy-based methods are available to those skilled in the art.

FIG. 3 is a flow diagram 300 of a method for determining voiced and unvoiced speech using an accelerometer-based VAD, under an embodiment. Generally, the energy is calculated by defining a standard window size over which the calculation is to take place and summing the square of the amplitude over time as

$$Energy = {\sum }_{i} {x}_{i}^{2} ,$$

where i is the digital sample subscript and ranges from the beginning of the window to the end of the window.

Referring to FIG. 3, operation begins upon receiving accelerometer data, at block 302. The processing associated with the VAD includes filtering the data from the accelerometer to preclude aliasing, and digitizing the filtered data for processing, at block 304. The digitized data is segmented into windows 20 milliseconds (msec) in length, and the data is stepped 8 msec at a time, at block 306. The processing further includes filtering the windowed data, at block 308, to remove spectral information that is corrupted by noise or is otherwise unwanted. The energy in each window is calculated by summing the squares of the amplitudes as described above, at block 310. The calculated energy values can be normalized by dividing the energy values by the window length; however, this involves an extra calculation and is not needed as long as the window length is not varied.

The calculated, or normalized, energy values are compared to a threshold, at block 312. The speech corresponding to the accelerometer data is designated as voiced speech when the energy of the accelerometer data is at or above a threshold value, at block 314. Likewise, the speech corresponding to the accelerometer data is designated as unvoiced speech when the energy of the accelerometer data is below the threshold value, at block 316. Noise suppression systems of alternative embodiments can use multiple threshold values to indicate the relative strength or confidence of the voicing signal, but are not so limited. Multiple subbands may also be processed for increased accuracy.

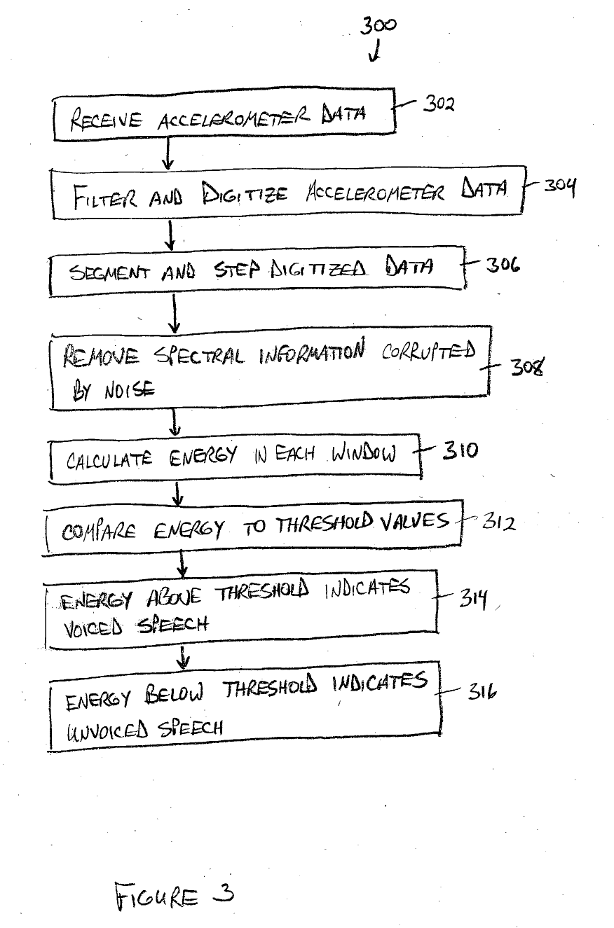

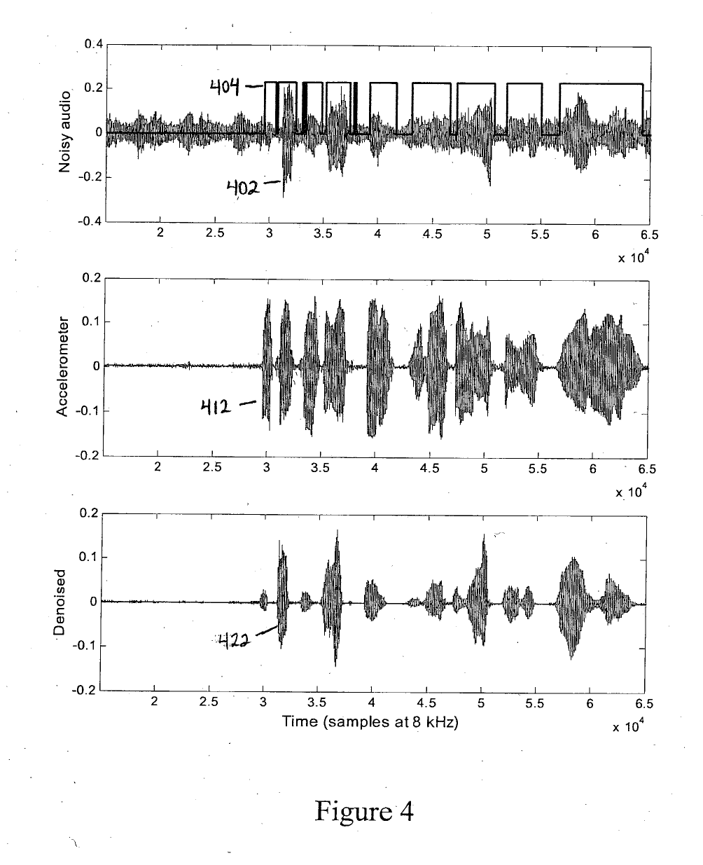

FIG. 4 shows plots including a noisy audio signal (live recording) 402 along with a corresponding accelerometer-based VAD signal 404, the corresponding accelerometer output signal 412, and the denoised audio signal 422 following processing by the Pathfinder system using the VAD signal 404, under an embodiment. In this example, the accelerometer data has been bandpass filtered between 500 and 2500 Hz to remove unwanted acoustic noise that can couple to the accelerometer below 500 Hz. The audio signal 402 was recorded using an Aliph microphone set and standard accelerometer in a babble noise environment inside a chamber measuring six (6) feet on a side and having a ceiling height of eight (8) feet. The Pathfinder system is implemented in real-time, with a delay of approximately 10 msec. The difference in the raw audio signal 402 and the denoised audio signal 422 shows noise suppression approximately in the range of 25-30 dB with little distortion of the desired speech signal. Thus, denoising using the accelerometer-based VAD information is effective.

Skin Surface Microphone (SSM) VAD Devices/Methods

Referring again to FIG. 1 and FIG. 1A, a VAD system 102A of an embodiment includes a SSM VAD device 130 providing data to an associated algorithm 140. The SSM is a conventional microphone modified to prevent airborne acoustic information from coupling with the microphone's detecting elements. A layer of silicone gel or other covering changes the impedance of the microphone and prevents airborne acoustic information from being detected to a significant degree. Thus this microphone is shielded from airborne acoustic energy but is able to detect acoustic waves traveling in media other than air as long as it maintains physical contact with the media. In order to efficiently detect acoustic energy in human skin, then, the gel is matched to the mechanical impedance properties of the skin.

During speech, when the SSM is placed on the cheek or neck, vibrations associated with speech production are easily detected. However, the airborne acoustic data is not significantly detected by the SSM. The tissue-borne acoustic signal, upon detection by the SSM, is used to generate the VAD signal in processing and denoising the signal of interest, as described above with reference to the energy/threshold method used with accelerometer-based VAD signal and FIG. 3.

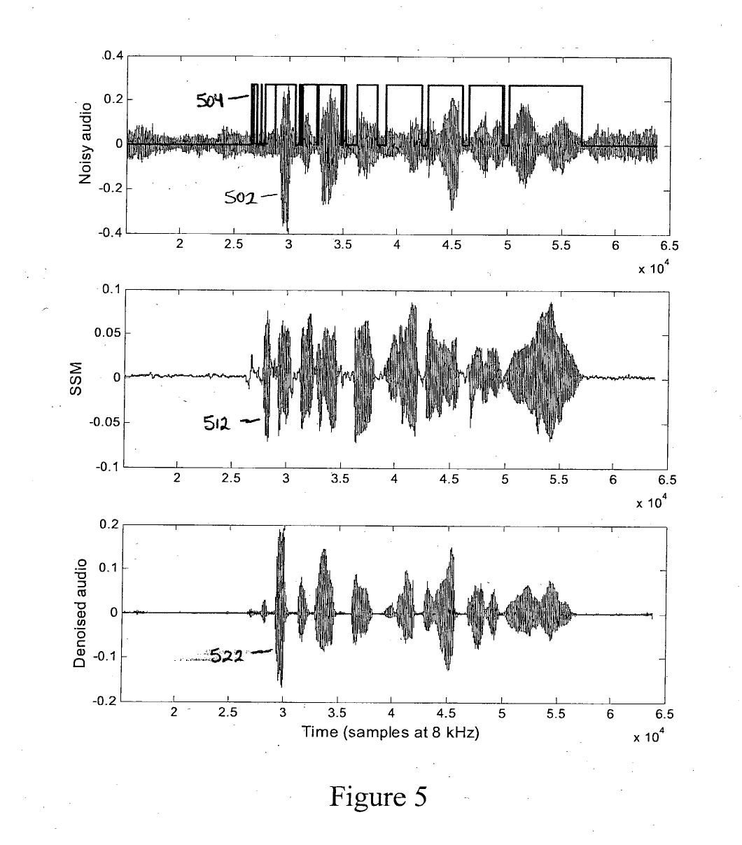

FIG. 5 shows plots including a noisy audio signal (live recording) 502 along with a corresponding SSM-based VAD signal 504, the corresponding SSM output signal 512, and the denoised audio signal 522 following processing by the Pathfinder system using the VAD signal 504, under an embodiment. The audio signal 502 was recorded using an Aliph microphone set and standard accelerometer in a babble noise environment inside a chamber measuring six (6) feet on a side and having a ceiling height of eight (8) feet. The Pathfinder system is implemented in real-time, with a delay of approximately 10 msec. The difference in the raw audio signal 502 and the denoised audio signal 522 clearly show noise suppression approximately in the range of 20-25 dB with little distortion of the desired speech signal. Thus, denoising using the SSM-based VAD information is effective.

Electromagnetic (EM) Vibrometer VAD Devices/Methods

Returning to FIG. 1 and FIG. 1A, a VAD system 102A of an embodiment includes an EM vibrometer VAD device 130 providing data to an associated algorithm 140. The EM vibrometer devices also detect tissue vibration, but can do so at a distance and without direct contact of the tissue targeted for measurement. Further, some EM vibrometer devices can detect vibrations of internal tissue of the human body. The EM vibrometers are unaffected by acoustic noise, making them good choices for use in high noise environments. The Pathfinder system of an embodiment receives VAD information from EM vibrometers including, but not limited to, RF vibrometers and laser vibrometers, each of which are described in turn below.

The RF vibrometer operates in the radio to microwave portion of the electromagnetic spectrum, and is capable of measuring the relative motion of internal human tissue associated with speech production. The internal human tissue includes tissue of the trachea, cheek, jaw, and/or nose/nasal passages, but is not so limited. The RF vibrometer senses movement using low-power radio waves, and data from these devices has been shown to correspond very well with calibrated targets. As a result of the absence of acoustic noise in the RF vibrometer signal, the VAD system of an embodiment uses signals from these devices to construct a VAD using the energy/threshold method described above with reference to the accelerometer-based VAD and FIG. 3.

An example of an RF vibrometer is the General Electromagnetic Motion Sensor (GEMS) radiovibrometer available from Aliph, San Francisco, Calif. Other RF vibrometers are described in the Related Applications and by Gregory C. Burnett in “The Physiological Basis of Glottal Electromagnetic Micropower Sensors (GEMS) and Their Use in Defining an Excitation Function for the Human Vocal Tract”, Ph.D. Thesis, University of California Davis, January 1999.

Laser vibrometers operate at or near the visible frequencies of light, and are therefore restricted to surface vibration detection only, similar to the accelerometer and the SSM described above. Like the RF vibrometer, there is no acoustic noise associated with the signal of the laser vibrometers. Therefore, the VAD system of an embodiment uses signals from these devices to construct a VAD using the energy/threshold method described above with reference to the accelerometer-based VAD and FIG. 3.

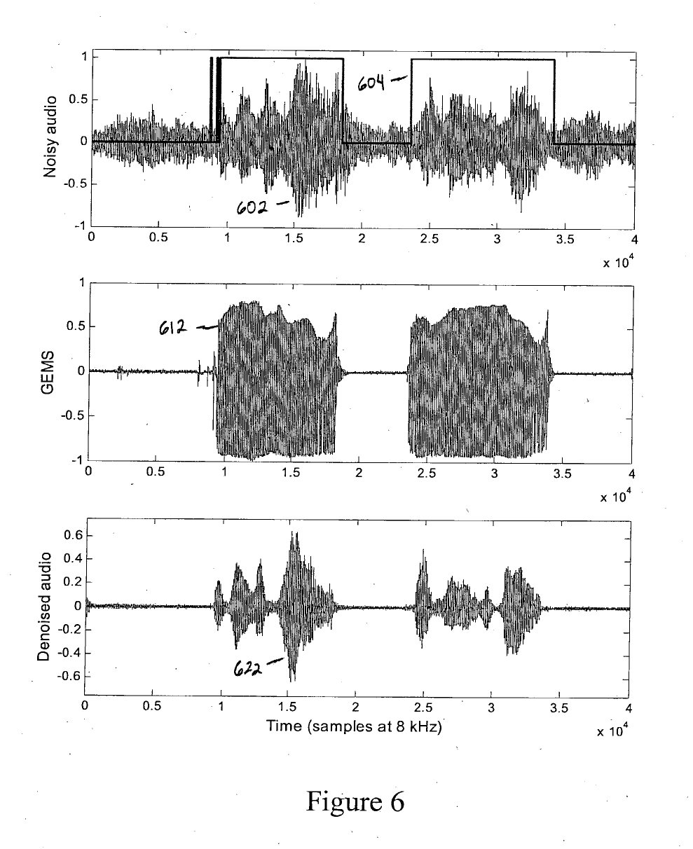

FIG. 6 shows plots including a noisy audio signal (live recording) 602 along with a corresponding GEMS-based VAD signal 604, the corresponding GEMS output signal 612, and the denoised audio signal 622 following processing by the Pathfinder system using the VAD signal 604, under an embodiment. The GEMS-based VAD signal 604 was received from a trachea-mounted GEMS radiovibrometer from Aliph, San Francisco, Calif. The audio signal 602 was recorded using an Aliph microphone set in a babble noise environment inside a chamber measuring six (6) feet on a side and having a ceiling height of eight (8) feet. The Pathfinder system is implemented in real-time, with a delay of approximately 10 msec. The difference in the raw audio signal 602 and the denoised audio signal 622 clearly show noise suppression approximately in the range of 20-25 dB with little distortion of the desired speech signal. Thus, denoising using the GEMS-based VAD information is effective. It is clear that both the VAD signal and the denoising are effective, even though the GEMS is not detecting unvoiced speech. Unvoiced speech is normally low enough in energy that it does not significantly affect the convergence of H₁(z) and therefore the quality of the denoised speech.

Direct Glottal Motion Measurement VAD Devices/Methods

Referring to FIG. 1 and FIG. 1A, a VAD system 102A of an embodiment includes a direct glottal motion measurement VAD device 130 providing data to an associated algorithm 140. Direct Glottal Motion Measurement VAD devices of the Pathfinder system of an embodiment include the Electroglottograph (EGG), as well as any devices that directly measure vocal fold movement or position. The EGG returns a signal corresponding to vocal fold contact area using two or more electrodes placed on the sides of the thyroid cartilage. A small amount of alternating current is transmitted from one or more electrodes, through the neck tissue (including the vocal folds) and over to other electrode(s) on the other side of the neck. If the folds are touching one another then the amount of current flowing from one set of electrodes to another is increased; if they are not touching the amount of current flowing is decreased. As with both the EM vibrometer and the SSM, there is no acoustic noise associated with the signal of the EGG. Therefore, the VAD system of an embodiment uses signals from the EGG to construct a VAD using the energy/threshold method described above with reference to the accelerometer-based VAD and FIG. 3.

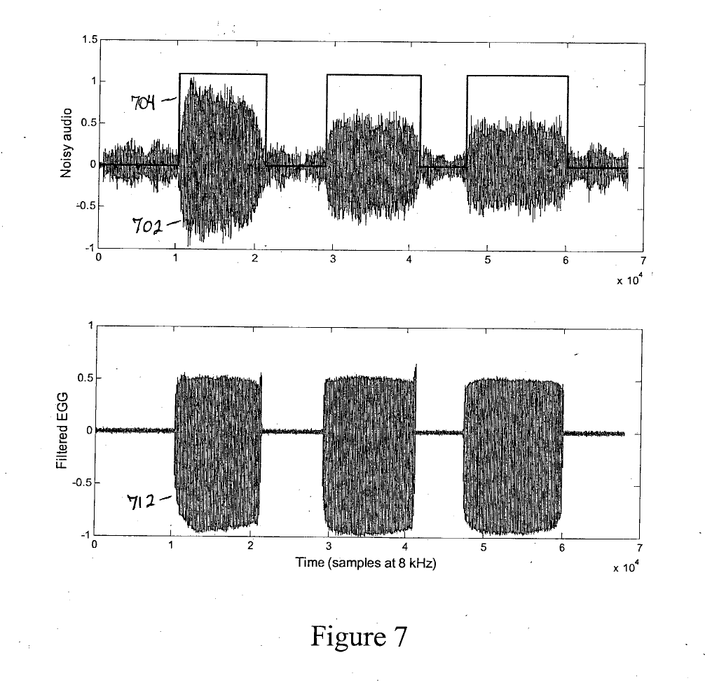

FIG. 7 shows plots including recorded acoustic data 702 spoken by an English-speaking male with digitally added noise along with a corresponding EGG-based VAD signal 704, and the corresponding highpass filtered EGG output signal 712, under an embodiment. A comparison of the acoustic data 702 and the EGG output signal shows the EGG to be accurate at detecting voiced speech, although the EGG cannot detect unvoiced speech or very soft voiced speech in which the vocal folds are not touching. In experiments, though, the inability to detect unvoiced and softly voiced speech (which are both very low in energy) has not significantly affected the ability of the system to denoise speech under normal environmental conditions. More information on the EGG is provided by D. G. Childers and A. K. Krishnamurthy in “A Critical Review of Electroglottography”, CRC Crit Rev Biomedical Engineering, 12, pp. 131-161, 1985.

Video detection VAD Devices/Methods

The VAD system 102A of an embodiment, with reference to FIG. 1 and FIG. 1A, includes a video detection VAD device 130 providing data to an associated algorithm 140. A video camera and processing system of an embodiment detect movement of the vocal articulators including the jaw, lips, teeth, and tongue. Video and computer systems currently under development support computer vision in three dimensions, thus enabling a video-based VAD. Information about the tools to build such systems is available at http://www.intel.com/research/mrl/research/opencv/.

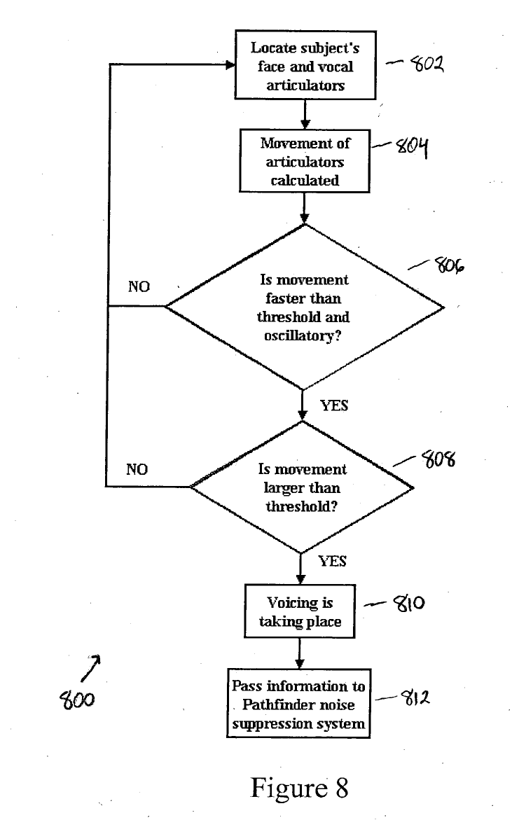

The Pathfinder system of an embodiment can use components of a video system to detect the motion of the articulators and generate VAD information. FIG. 8 is a flow diagram 800 of a method for determining voiced speech using a video-based VAD, under an embodiment. Components of the video system locate a user's face and vocal articulators, at block 802, and calculate movement of the articulators, at block 804. Components of the video system and/or the Pathfinder system determine if the calculated movement of the articulators is faster than a threshold speed and oscillatory (moving back and forth and distinguishable from simple translational motion), at block 806. If the movement is slower than the threshold speed and/or not oscillatory, operation continues at block 802 as described above.

When the movement is faster than the threshold speed and oscillatory, as determined at block 806, the components of the video system and/or the Pathfinder system determine if the movement is larger than a threshold value, at block 808. If the movement is less than the threshold value, operation continues at block 802 as described above. When the movement is larger than the threshold value, the components of the video VAD system determine that voicing is taking place, at block 810, and transfer the associated VAD information to the Pathfinder system, at block 812. This video-based VAD would be immune to the affects of acoustic noise, and could be performed at a distance from the user or speaker, making it particularly useful for surveillance operations.

Acoustic Information-Based VAD Devices/Methods

As described above with reference to FIG. 1 and FIG. 1B, when using the VAD with a noise suppression system, the VAD signal is processed independently of the noise suppression system, so that the receipt and processing of VAD information is independent from the processing associated with the noise suppression. The acoustic information-based VAD devices attain this independence through processing in that they may use the same hardware to receive signals into the noise suppression system while using independent techniques (software, algorithms, routines) to process the received signals. In some cases, however, acoustic microphones may be used for VAD construction but not noise suppression.

The acoustic information-based VAD devices/methods of an embodiment rely on one or more conventional acoustic microphones to detect the speech of interest. As such, they are more susceptible to environmental acoustic noise and generally do not operate reliably in all noise environments. However, the acoustic information-based VAD has the advantage of being simpler, cheaper, and being able to use the same microphones for both the VAD and the acoustic data microphones. Therefore, for some applications where cost is more important than high-noise performance, these VAD solutions may be preferable. The acoustic information-based VAD devices/methods of an embodiment include, but are not limited to, single microphone VAD, Pathfinder VAD, stereo VAD (SVAD), array VAD (AVAD), and other single-microphone conventional VAD devices/methods, as described below.

Single Microphone VAD Devices/Methods

This is probably the simplest way to detect that a user is speaking. Referring to FIG. 1 and FIG. 1B, a VAD system 102B of an embodiment includes a VAD algorithm 150 that receives data 164 from a single microphone of the corresponding signal processing system 100. The microphone (normally a “close-talk” (or gradient) microphone) is placed very close to the mouth of the user, sometimes in direct contact with the lips. A gradient microphone is relatively insensitive to sound originating more than a few centimeters from the microphone (for a range of frequencies, normally below 1 kHz) and so the gradient microphone signals generally have a relatively high SNR. Of course, the performance realized from the single microphone depends on the distance between the mouth of the user and the microphone, the severity of the environmental noise, and the user's willingness to place something so close to his or her lips. Because at least part of the spectrum of the recorded data or signal from the closely-placed single microphone typically has a relatively high SNR, the Pathfinder system of an embodiment can use signals from the single microphone to construct a VAD using the energy/threshold method described above with reference to the accelerometer-based VAD and FIG. 3.

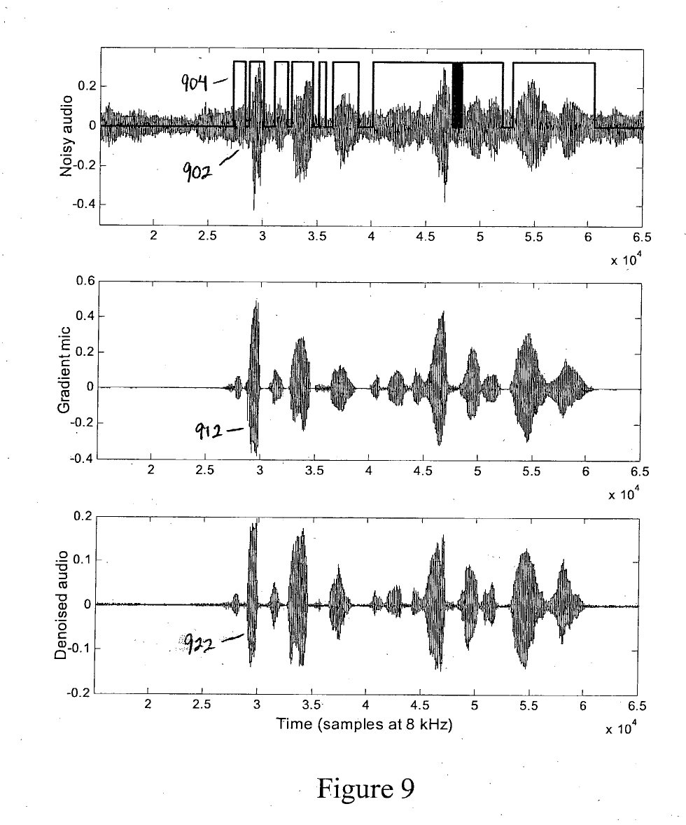

FIG. 9 shows plots including a noisy audio signal (live recording) 902 along with a corresponding single (gradient) microphone-based VAD signal 904, the corresponding gradient microphone output signal 912, and the denoised audio signal 922 following processing by the Pathfinder system using the VAD signal 904, under an embodiment. The audio signal 902 was recorded using an Aliph microphone set in a babble noise environment inside a chamber measuring six (6) feet on a side and having a ceiling height of eight (8) feet. The Pathfinder system is implemented in real-time, with a delay of approximately 10 msec. The difference in the raw audio signal 902 and the denoised audio signal 922 shows noise suppression approximately in the range of 25-30 dB with little distortion of the desired speech signal. While these results show that the single microphone-based VAD information can be effective.

Pathfinder VAD (PVAD) Devices/Methods

Returning again to FIG. 1 and FIG. 1B, a PVAD system 102B of an embodiment includes a PVAD algorithm 150 that receives data 164 from a microphone array of the corresponding signal processing system 100. The microphone array includes two microphones, but is not so limited. The PVAD of an embodiment operates in the time domain and locates the two microphones of the microphone array within a few centimeters of each other. At least one of the microphones is a directional microphone.

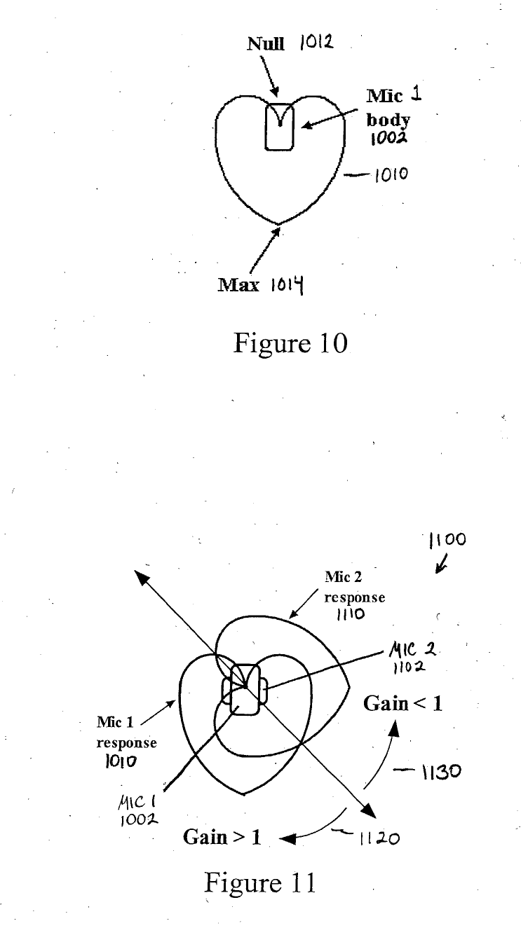

FIG. 10 shows a single cardioid unidirectional microphone 1002 of the microphone array, along with the associated spatial response curve 1010, under an embodiment. The unidirectional microphone 1002, also referred to herein as the speech microphone 1002, or MIC 1, is oriented so that the mouth of the user is at or near a maximum 1014 in the spatial response 1010 of the speech microphone 1002. This system is not, however, limited to cardiod directional microphones.

FIG. 11 shows a microphone array 1100 of a PVAD system, under an embodiment. The microphone array 1100 includes two cardioid unidirectional microphones MIC 1 1002 and MIC 2 1102, each having a spatial response curve 1010 and 1110, respectively. When used in the microphone array 1100, there is no restriction on the type of microphone used as the speech microphone MIC 1; however, best performance is realized when the speech microphone MIC 1 is a unidirectional microphone and oriented such that the mouth of the user is at or near a maximum in the spatial response curve 1010. This ensures that the difference in the microphone signals is large when speech is occurring.

One embodiment of the microphone configuration including MIC 1 and MIC 2 places the microphones near the user's ear. The configuration orients the speech microphone MIC 1 toward the mouth of the user, and orients the noise microphone MIC 2 away from the head of the user, so that the maximums of each microphone's spatial response curve are displaced approximately 90 degrees from each other. This allows the noise microphone MIC 2 to sufficiently capture noise from the front of the head while at the same time not capturing too much speech from the user.

Two alternative embodiments of the microphone configuration orient the microphones 1102 and 1002 so that the maximums of each microphone's spatial response curve are displaced approximately 75 degrees and 135 degrees from each other, respectively. These configurations of the PVAD system place the microphones as close together as possible to simplify the H₁(z) calculation, and orient the microphones in such a way that the speech microphone MIC 1 is detecting mostly speech and the noise microphone MIC 2 is detecting mostly noise (i.e., H₂(z) is relatively small). The displacements between the maximums of each microphone's spatial response curve can be up to approximately 180 degrees, but should not be less than approximately 45 degrees.

The PVAD system uses the Pathfinder method of calculating the differential path between the speech microphone and the noise microphone (known in Pathfinder as H₁, as described herein) to assist in calculating the VAD. Instead of using this information for noise suppression, the VAD system uses the gain of H₁ to decide when to denoise. Examining the ratio of the energy of the signal in the speech microphone to that in the noise microphone, a PVAD H₁ gain (referred to herein as gain) is calculated as

$$Gain = \vert {H}_{1} ( z ) \vert = \frac{Energy of speech mic}{Energy of noise mic} = \frac{{\sum }_{i} {x}_{i}^{2}}{{\sum }_{i} {y}_{i}^{2}} ,$$

where xᵢ is the i^{th} sample of the digitized signal of the speech microphone, and yᵢ is the i^{th} sample of the digitized signal of the noise microphone. There is no requirement to calculate H₁ adaptively for this VAD application. Although this example is in the digital domain, the results are valid in the analog domain as well. The gain can be calculated in either the time or frequency domain as well. In the frequency domain, the gain parameter is the sum of the squares of the H₁ coefficients. As above, the length of the window is not included in the energy calculation because when calculating the ratio of the energies the length of the window of interest cancels out. Finally, this example is for a single frequency subband, but is valid for any number of desired subbands.

Referring again to FIG. 11, the spatial response curves 1010 and 1110 for the microphone array 1100 show gain greater than unity in a first hemisphere 1120 and gain less than unity in a second hemisphere 1130, but are not so limited. This, along with the relative proximity of the speech microphone MIC 1 to the mouth of the user, helps in differentiating speech from noise.

The microphone array 1100 of the PVAD embodiment provides additional benefits in that it is conducive to optimal performance of the Pathfinder system while allowing the same two microphones to be used for VAD and for denoising, thereby reducing system cost. For optimal performance of the VAD, though, the two microphones are oriented in opposite directions to take advantage of the very large change in gain for that configuration.

The PVAD of an alternative embodiment includes a third unidirectional microphone MIC 3 (not shown), but is not so limited. The third microphone MIC 3 is oriented opposite to MIC 1 and is used for VAD only, while MIC 2 is used for noise suppression only, and MIC 1 is used for both VAD and noise suppression. This results in better overall system performance at the cost of an additional microphone and the processing of 50% more acoustic data.

The Pathfinder system of an embodiment uses signals from the PVAD to construct a VAD using the energy/threshold method described above with reference to the accelerometer-based VAD and FIG. 3. Because there can be a significant amount of noise in the microphone data, however, it is not always possible to use the energy/threshold VAD detection algorithm of the accelerometer-based VAD embodiment. An alternative VAD embodiment uses past values of the gain (during noise-only times) to determine if voicing is occurring, as described below.

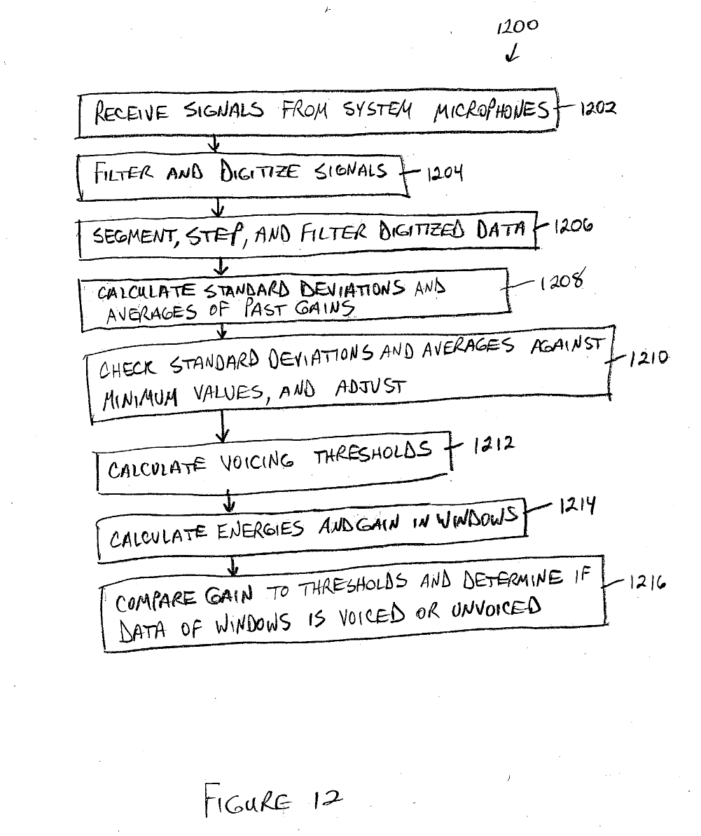

FIG. 12 is a flow diagram 1200 of a method for determining voiced and unvoiced speech using gain values, under an alternative embodiment of the PVAD. Operation begins with the receiving of signals via the system microphones, at block 1202. Components of the PVAD system filter the data to preclude aliasing, and digitize the filtered data, at block 1204. The digitized data from the microphones is segmented into windows 20 msec in length, and the data is stepped 8 msec at a time, at block 1206. Further, the windowed data is filtered to remove unwanted spectral information. The standard deviation (SD) of the last approximately 50 gain calculations from noise-only windows (vector OLD_STD) is calculated, along with the average (AVE) of OLD_STD, at block 1208, but the embodiment is not so limited. The values for AVE and SD are compared against prespecified minimum values and, if less than the minimum values, are increased to the minimum values, respectively, at block 1210.

The components of the PVAD system next calculate voicing thresholds by summing the AVE with a multiple of the SD, at block 1212. A lower threshold results from summing the AVE plus 1.5 times the SD, while an upper threshold results from summing the AVE plus 4 times the SD. The energy in each window is calculated by summing the squares of the amplitudes, at block 1214. Further, at block 1214, the gain is computed by taking the ratio of the energy in MIC 1 to the energy in MIC 2. A small cutoff value is added to the MIC 2 energy to ensure stability, but the embodiment is not so limited.

The calculated gains are compared to the thresholds, at block 1216, with three possible outcomes. When the gain is less than the lower threshold, a determination is made that the window does not include voiced speech, and the OLD_STD vector is updated with the new gain value. When the gain is greater than the lower threshold and less than the upper threshold, a determination is made that the window does not include voiced speech, but the speech is suspected of being voiced speech, and the OLD_STD vector is not updated with the new gain value. When the gain is greater than both the lower and upper thresholds, a determination is made that the window includes voiced speech, and the OLD_STD vector is not updated with the new gain value.

Regardless of the implementation of this method, the idea is to use the larger gain of H₁(z)=M₁(z)/M₂(z) when speech is occurring to differentiate it from the noisy background. The gain calculated during speech should be larger, since, due to the microphone configuration, the speech is much louder in the speech microphone (MIC 1) than it is in the noise microphone (MIC 2). Conversely, the noise is often more geometrically diffuse, and will often be louder in MIC 2 than in MIC 1. This is not always true if an omnidirectional microphone is used as the speech microphone, which may limit the level of the noise in which the system can operate.

Note that an acoustic-only method of denoising is more susceptible to environmental noise. However, tests have shown that the unidirectional-unidirectional microphone configuration described above provides satisfactory results with SNRs in MIC 1 of slightly less than 0 dB. Thus, this PVAD-based noise suppression system can operate effectively in almost all noise environments that a user is likely to encounter. Also, if needed, an increase in the SNR of MIC 1 can be realized by moving the microphones closer to the user's mouth.

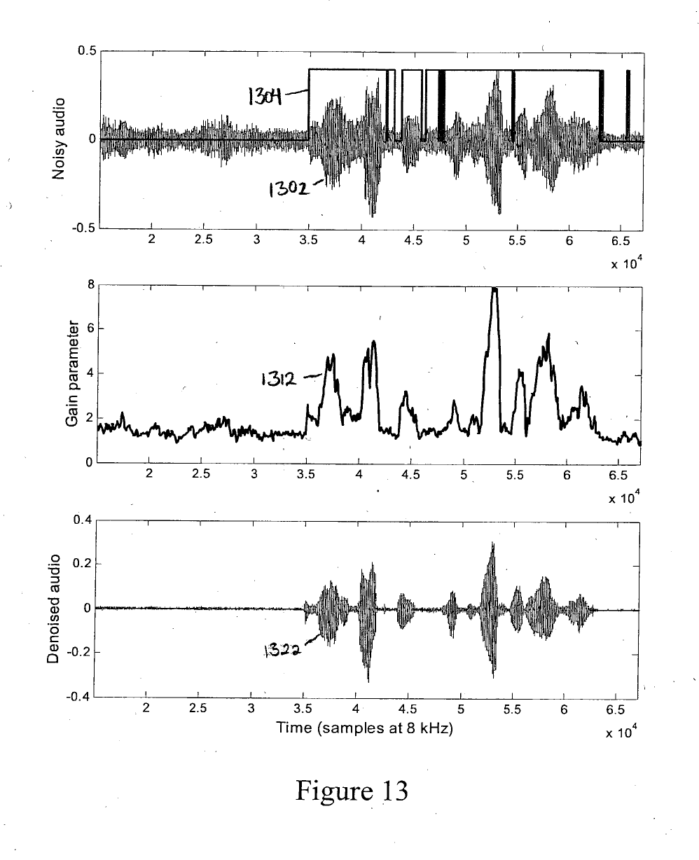

FIG. 13 shows plots including a noisy audio signal (live recording) 1302 along with a corresponding microphone-based PVAD signal 1304, the corresponding PVAD gain signal 1312, and the denoised audio signal 1322 following processing by the Pathfinder system using the PVAD signal 1304, under an embodiment. The audio signal 1302 was recorded using an Aliph microphone set in a babble noise environment inside a chamber measuring six (6) feet on a side and having a ceiling height of eight (8) feet. The Pathfinder system is implemented in real-time, with a delay of approximately 10 msec. The difference in the raw audio signal 1302 and the denoised audio signal 1322 shows noise suppression approximately in the range of 20-25 dB with little distortion of the desired speech signal. Thus, denoising using the microphone-based PVAD information is effective.

Stereo VAD (SVAD) Devices/Methods

Referring to FIG. 1 and FIG. 1B, an SVAD system 102B of an embodiment includes an SVAD algorithm 150 that receives data 164 from a frequency-based two-microphone array of the corresponding signal processing system 100. The SVAD algorithm operates on the theory that the frequency spectrum of the received speech allows it to be discemable from noise. As such, the processing associated with the SVAD devices/methods includes a comparison of average FFTs between microphones. The SVAD uses two microphones in an orientation similar to the PVAD described above and with reference to FIG. 11, and also depends on noise data from previous windows to determine whether the present window contains speech. As described above with the PVAD devices/methods, the speech microphone is referred to herein as MIC 1 and the noise microphone referred to as MIC 2.

Referring to FIG. 1, the Pathfinder noise suppression system uses two microphones to characterize the speech (MIC 1) and the noise (MIC 2). Naturally, there is a mixture of speech and noise in both microphones, but it is assumed that the SNR of MIC 1 is greater than that of MIC 2. This generally means that MIC 1 is closer or better oriented with respect to the speech source (the user) than MIC 2, and that any noise sources are located farther away from MIC 1 and MIC 2 than the speech source. However, the same effect can be accomplished by using a combination of omnidirectional and unidirectional or similar microphones.

The difference in SNR between the two microphones can be exploited in either the time domain or the frequency domain. In order to separate the noise from the speech, it is necessary to calculate the average spectrum of the noise over time. This is accomplished using an exponential averaging method as

L(i, k)=αL(i−1,k)+(1−α)S(i,k),

where α controls the smoothness of the averaging (0.999 results in a very smoothed average, 0.9 is not very smooth). The variables L(i,k) and S(i,k) are the averaged and instantaneous variables, respectively, i represents the discrete time sample, and k represents the frequency bin, the number of which is determined by the length of the FFT. Conventional averaging or a moving average can also be used to determine these values.

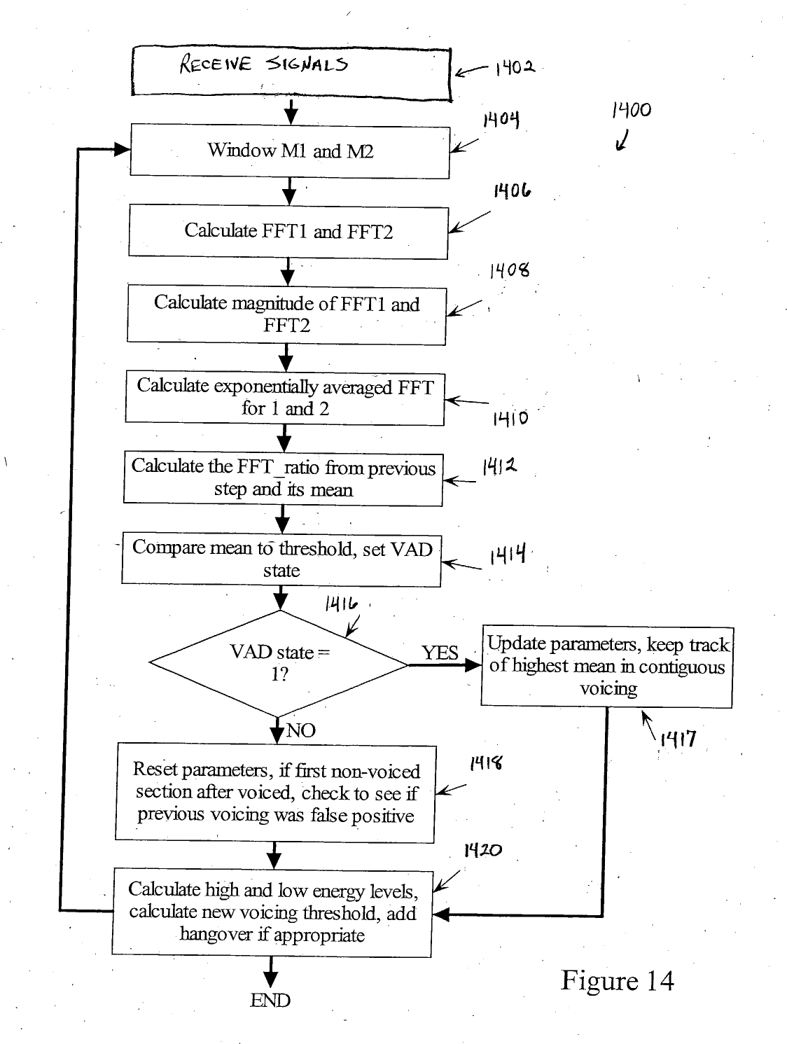

FIG. 14 is a flow diagram 1400 of a method for determining voiced and unvoiced speech using a stereo VAD, under an embodiment. In this example, data was recorded at 8 kHz (taking proper precautions to preclude aliasing) using two microphones, as described with reference to FIG. 1. The windows used were 20 milliseconds long with an 8 millisecond step.

Operation begins upon receiving signals at the two microphones, at block 1402. Data from the microphone signals are properly filtered to preclude aliasing, and are digitized for processing. Further, the previous 160 samples from MIC 1 and MIC 2 are windowed using a Hamming window, at block 1404. Components of the SVAD system compute the magnitude of the FFTs of the windowed data to get FFT1 and FFT2, at blocks 1406 and 1408.

Using the exponential averaging method described above along with an α value of 0.85, FFT1 and FFT2 are exponentially averaged to generate MF1 and MF2, at block 1410. Using MF1 and MF2, at block 1412, the system computes the VAD_det as the mean of the ratio of MF 1 and MF2 with a cutoff, as

$${VAD\_det}_{i} = \frac{1}{128} {\sum }_{k} ( \frac{{MF1}_{i , k}}{{MF2}_{i , k} + cutoff} )$$

where i is now the window of interest, k is the frequency bin, and the cutoff keeps the ratio reasonably sized when the MIC 2 frequency bin amplitude is very small. Because the FFTs are of length 128, divide the result by 128 to get the average value of the ratio.

Components of the Pathfinder system compare the determinant VAD_det to the voicing threshold V_thresh, at block 1414. Further, and in response to the comparison, components of the system set VAD_state to zero if the value of VAD_det is below V_thresh, and set VAD_state to one if the value of VAD_det is above V_thresh.

A determination is made as to whether the VAD_state equals one, at block 1416. When the VAD_state equals one, components of the Pathfinder system update parameters along with a counter of the contiguous voicing section that records the largest value of the VAD_det, at block 1417, and operation continues at block 1420 as described below. If an unvoiced window appears after a voiced one, the record of the largest VAD_det in the previous contiguous voiced section (which can include one or more windows) is examined to see if the voicing indication was in error. If the largest VAD_det in the section is below a set threshold (the low determinant level plus 40% of the difference between the low and high determinant levels, for example) the voicing state is set to a value of negative one (−1) for that window. This can be used to alert the denoising algorithm that the previous voiced section was in fact unlikely to be voiced so that the Pathfinder system can amend its coefficient calculations.

When the SVAD system determines the VAD_state equals zero, at block 1416, components of the SVAD system reset parameters including the largest VAD_det, at block 1418. Also, if the previous window was voiced, a check is performed to determine whether the previous voiced section was a false positive. Components of the Pathfinder system then update high and low determinant levels, which are used to calculate the voicing threshold V_thresh, at block 1420. Operation then returns to block 1402.

The low and high determinant levels in this embodiment are both calculated using exponential averaging, with the α values determined in response to whether the current VAD_det is above or below the low and high determinant levels, as follows. For the low determinant level, if the value of VAD_det is greater than the present low determinant level, the value of α is set equal to 0.999, otherwise 0.9 is used. For the high determinant level, a similar method is used, except that a is set equal to 0.999 when the current value of VAD_det is less than the current high determinant level, and α is set equal to 0.9 when the current value of VAD_det is greater than the current high determinant level. Conventional averaging or a moving average can be used to determine these levels in various alternative embodiments.

The threshold value of an embodiment is generally set to the low determinant level plus 15% of the difference between the low and high determinant levels, with an absolute minimum threshold also specified, but the embodiment is not so limited. The absolute minimum threshold should be set so that in quiet environments the VAD is not randomly triggered.

Alternative embodiments of the method for determining voiced and unvoiced speech using an SVAD can use different parameters, including window size, FFT size, cutoff value and α values, in performing a comparison of average FFTs between microphones. The SVAD devices/methods work with any kind of noise as long as the difference in the SNRs of the microphones is sufficient. The absolute SNR is not as much of a factor as the relative SNRs of the two microphones; thus, configuring the microphones to have a large relative SNR difference generally results in better VAD performance.

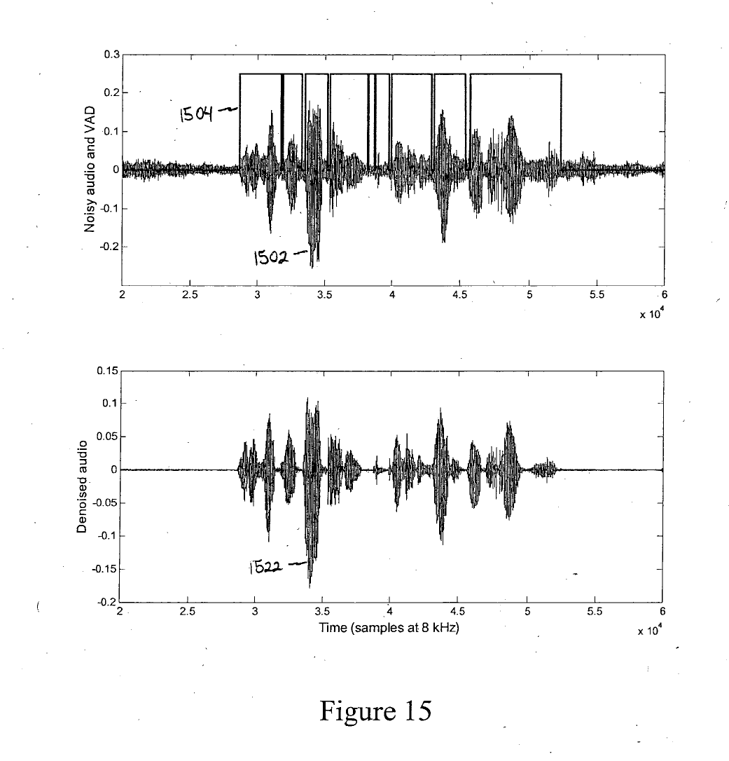

The SVAD devices/methods have been used successfully with a number of different microphone configurations, noise types, and noise levels. As an example, FIG. 15 shows plots including a noisy audio signal (live recording) 1502 along with a corresponding SVAD signal 1504, and the denoised audio signal 1522 following processing by the Pathfinder system using the SVAD signal 1504, under an embodiment. The audio signal 1502 was recorded using an Aliph microphone set in a babble noise environment inside a chamber measuring six (6) feet on a side and having a ceiling height of eight (8) feet. The Pathfinder system is implemented in real-time, with a delay of approximately 10 msec. The difference in the raw audio signal 1502 and the denoised audio signal 1522 shows noise suppression approximately in the range of 25-30 dB with little distortion of the desired speech signal when using the SVAD signal 1504.

Array VAD (AVAD) Devices/Methods

Referring to FIG. 1 and FIG. 1B, an AVAD system 102B of an embodiment includes an AVAD algorithm 150 that receives data 164 from a microphone array of the corresponding signal processing system 100. The microphone array of an AVAD-based system includes an array of two or more microphones that work to distinguish the speech of a user from environmental noise, but are not so limited. In one embodiment, two microphones are positioned a prespecified distance apart, thereby supporting accentuation of acoustic sources located in particular directions, such as on the axis of a line connecting the microphones, or on the midpoint of that line. An alternative embodiment uses beamforming or source tracking to locate the desired signal in the array's field of view and construct a VAD signal for use by an associated adaptive noise suppression system such as the Pathfinder system. Additional alternatives might be obvious to those skilled in the art when applying information like, for example, that found in “Microphone Arrays” by M. Brandstein and D. Ward, 2001, ISBN 3-540-41953-5.

The AVAD of an embodiment includes a two-microphone array constructed using Panasonic unidirectional microphones. The unidirectionality of the microphones helps to limit the detection of acoustic sources to those acoustic sources located forward of, or in front of, the array. However, the use of unidirectional microphones is not required, especially if the array is to be mounted such that sound can only approach from one side, such as on a wall. A linear distance of approximately 30.5 centimeters (cm) separates the two microphones, and a low-noise amplifier amplifies the data from the microphones for recording on a personal computer (PC) using National Instruments' Labview 5.0, but the embodiment is not so limited. Using this array, components of the system record microphone data at 12 bits and 32 kHz, and digitally filter and decimate the data down to 16 kHz. Alternative embodiments can use significantly lower resolution (perhaps 8-bit) and sampling rates (down to a few kHz) along with adequate analog prefiltering because fidelity of the acoustic data is of little to no interest.

The signal source of interest (a human speaker) was located at a distance of approximately 30 cm away from the microphone array on the midline of the microphone array. This configuration provided a zero delay between MIC 1 and MIC 2 for the signal source of interest and a non-zero delay for all other sources. Alternative embodiments can use a number of alternative configurations, each supporting different delay values, as each delay defines an active area in which the source of interest can be located.

For this experiment, two loudspeakers provide noise signals, with one loudspeaker located at a distance of approximately 50 cm to the right of the microphone array and a second loudspeaker located at a distance of approximately 150 cm to the right of and behind the human speaker. Street noise and truck noise having an SNR approximately in the range of 2-5 dB was played through these loudspeakers. Further, some recordings were made with no additive noise for calibration purposes.

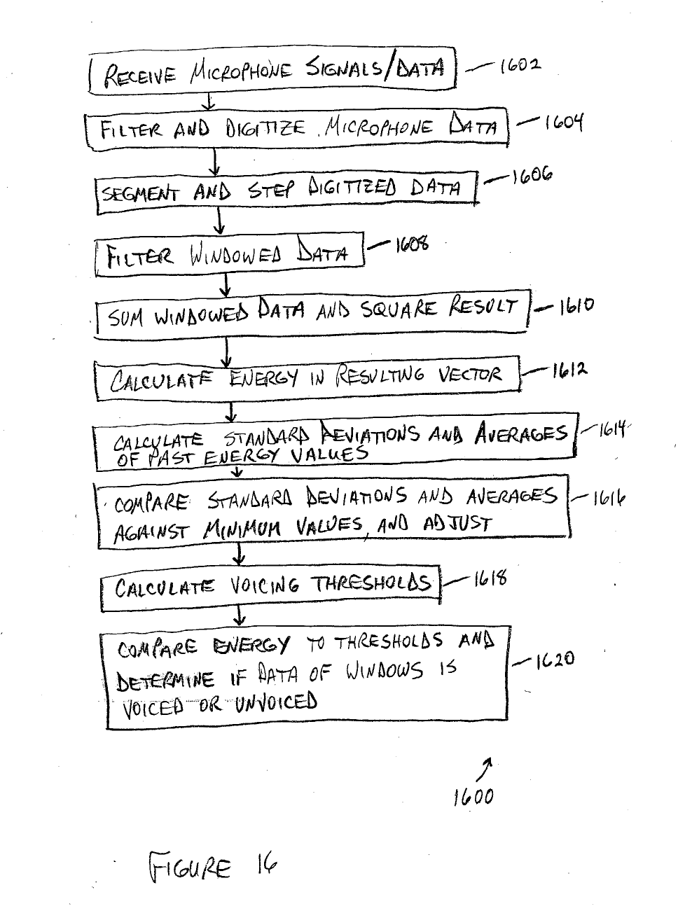

FIG. 16 is a flow diagram 1600 of a method for determining voiced and unvoiced speech using an AVAD, under an embodiment. Operation begins upon receiving signals at the two microphones, at block 1602. The processing associated with the VAD includes filtering the data from the microphones to preclude aliasing, and digitizing the filtered data for processing, at block 1604. The digitized data is segmented into windows 20 milliseconds (msec) in length, and the data is stepped 8 msec at a time, at block 1606. The processing further includes filtering the windowed data, at block 1608, to remove spectral information that is corrupted by noise or is otherwise unwanted.

The windowed data from MIC 1 is added to the windowed data from MIC 2, at block 1610, and the result is squared as

M₁₂=(M₁+M₂)².

The summing of the microphone data emphasizes the zero-delay elements of the resulting data. This constructively adds the portions of MIC 1 and MIC 2 that are in phase, and destructively adds the portions that are out of phase. Since the signal source of interest is in phase at all frequencies, it adds constructively, while the noise sources (whose phase relationships vary with frequency) generally add destructively. Then, the resulting signal is squared, greatly increasing the zero-delay elements. The resulting signal may use a simple energy/threshold algorithm to detect voicing (as described above with reference to the accelerometer-based VAD and FIG. 3), as the zero-delay elements have been substantially increased.

Continuing, the energy in the resulting vector is calculated by summing the squares of the amplitudes as described above, at block 1612. The standard deviation (SD) of the last 50 noise-only windows (vector OLD_STD) is calculated, along with the average (AVE) of OLD_STD, at block 1614. The values for AVE and SD are compared against prespecified minimum values and, if less than the minimum values, are increased to the minimum values, respectively, at block 1616.

The components of the Pathfinder system next calculate voicing thresholds by summing the AVE along with a multiple of the SD, at block 1618. A lower threshold results from summing the AVE plus 1.5 times the SD, while an upper threshold results from summing the AVE plus 4 times the SD. The energy is next compared to the thresholds, at block 1620, with three possible outcomes. When the energy is less than the lower threshold, a determination is made that the window does not include voiced speech, and the OLD_STD vector is updated with a new gain value. When the energy is greater than the lower threshold and less than the upper threshold, a determination is made that the window does not include voiced speech, but the speech is suspected of being voiced speech, and the OLD_STD vector is not updated with the new gain value. When the energy is greater than both the lower and upper thresholds, a determination is made that the window includes voiced speech, and the OLD_STD vector is not updated with the new gain value.

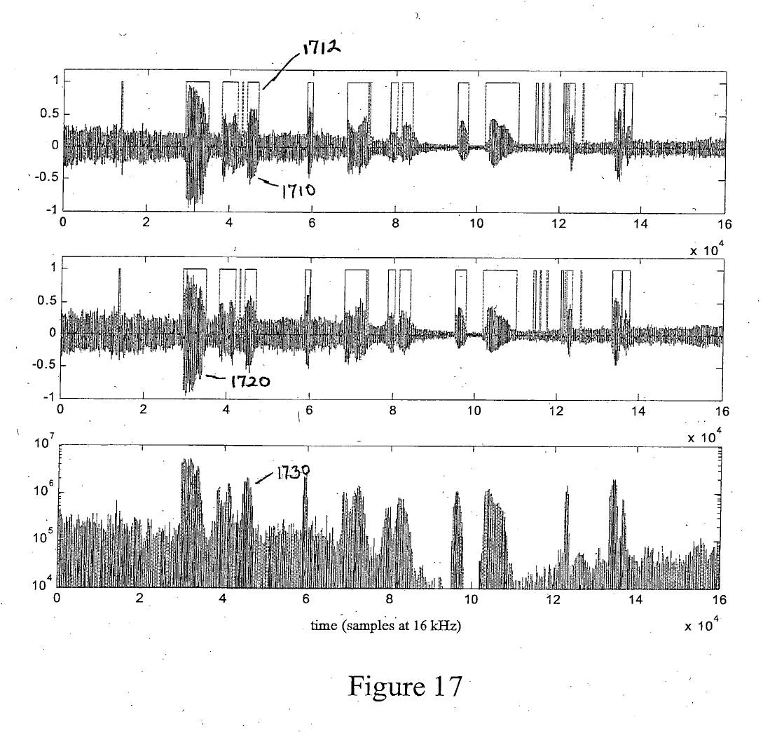

FIG. 17 shows plots including audio signals 1710 and 1720 from each microphone of an AVAD system along with corresponding VAD signals 1712 and 1722, respectively, under an embodiment. Also shown is the resulting signal 1730 generated from summing the audio signals 1710 and 1720. The speaker was located at a distance of approximately 30 cm from the midline of the microphone array, the noise used was truck noise, and the SNR was less than 0 dB at both microphones. The VAD signals 1712 and 1722 can be provided as inputs to the Pathfinder system or other noise suppression system.

Conventional Single-Microphone VAD Devices/Methods

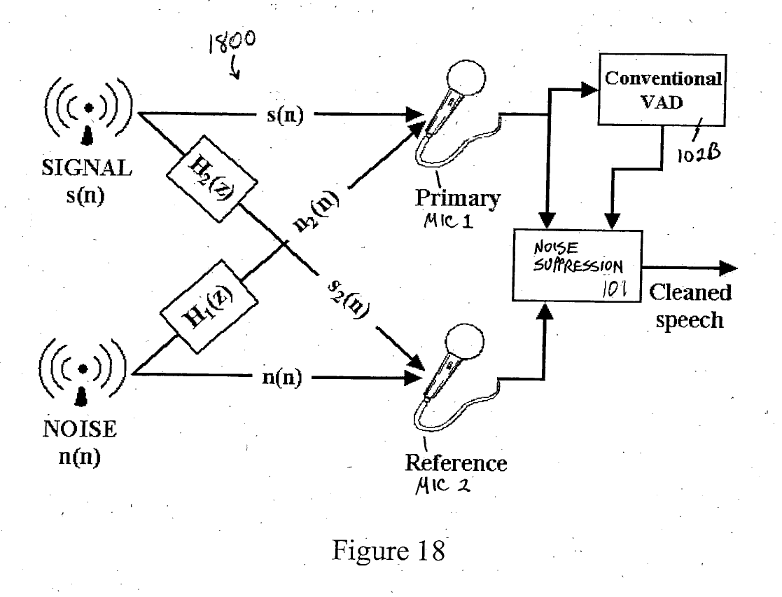

An embodiment of a noise suppression system uses signals of one microphone of a two-microphone system to generate VAD information, but is not so limited. FIG. 18 is a block diagram of a signal processing system 1800 including the Pathfinder noise suppression system 101 and a single-microphone VAD system 102B, under an embodiment. The system 1800 includes a primary microphone MIC 1, or speech microphone, and a reference microphone MIC 2, or noise microphone. The primary microphone MIC 1 couples signals to both the VAD system 102B and the Pathfinder system 101. The reference microphone MIC 2 couples signals to the Pathfinder system 101. Consequently, signals from the primary microphone MIC 1 provide speech and noise data to the Pathfinder system 101 and provide data to the VAD system 102B from which VAD information is derived.

The VAD system 102B includes a VAD algorithm, like those described in U.S. Pat. Nos. 4,811,404 and 5,687,243, to calculate a VAD signal, and the resultant information 104 is provided to the Pathfinder system 101, but the embodiment is not so limited. Signals received via the reference microphone MIC 2 of the system are used only for noise suppression.

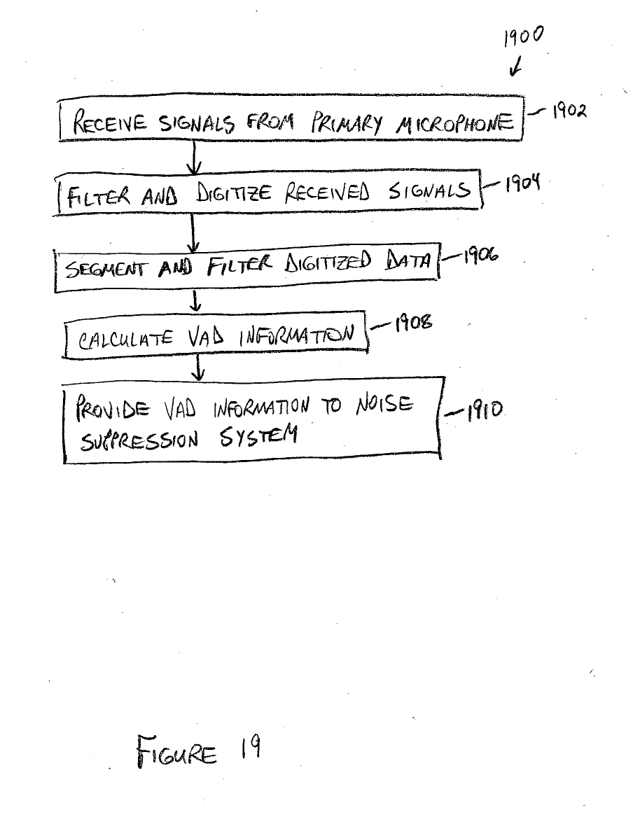

FIG. 19 is a flow diagram 1900 of a method for generating voicing information using a single-microphone VAD, under an embodiment. Operation begins upon receiving signals at the primary microphone, at block 1902. The processing associated with the VAD includes filtering the data from the primary microphone to preclude aliasing, and digitizing the filtered data for processing at an appropriate sampling rate (generally 8 kHz), at block 1904. The digitized data is segmented and filtered as appropriate to the conventional VAD, at block 1906. The VAD information is calculated by the VAD algorithm, at block 1908, and provided to the Pathfinder system for use in denoising operations, at block 1910.

Airflow-Derived VAD Devices/Methods

An airflow-based VAD device/method uses airflow from the mouth and/or nose of the user to construct a VAD signal. Airflow can be measured using any number of methods known in the art, and is separated from breathing and gross motion flow in order to yield accurate VAD information. Airflow is separated from breathing and gross motion flow by highpass filtering the flow data, as breathing and gross motion flow are composed of mostly low frequency (less than 100 Hz) energy. An example of a device for measuring airflow is Glottal Enterprise's Pneumotach Masks, and further information is available at http://www.glottal.com.

Using the airflow-based VAD device/method, the airflow is relatively free of acoustic noise because the airflow is detected very near the mouth and nose. As such, an energy/threshold algorithm can be used to detect voicing and generate a VAD signal, as described above with reference to the accelerometer-based VAD and FIG. 3. Alternative embodiments of the airflow-based VAD device and/or associated noise suppression system can use other energy-based methods to generate the VAD signal, as known to those skilled in the art.

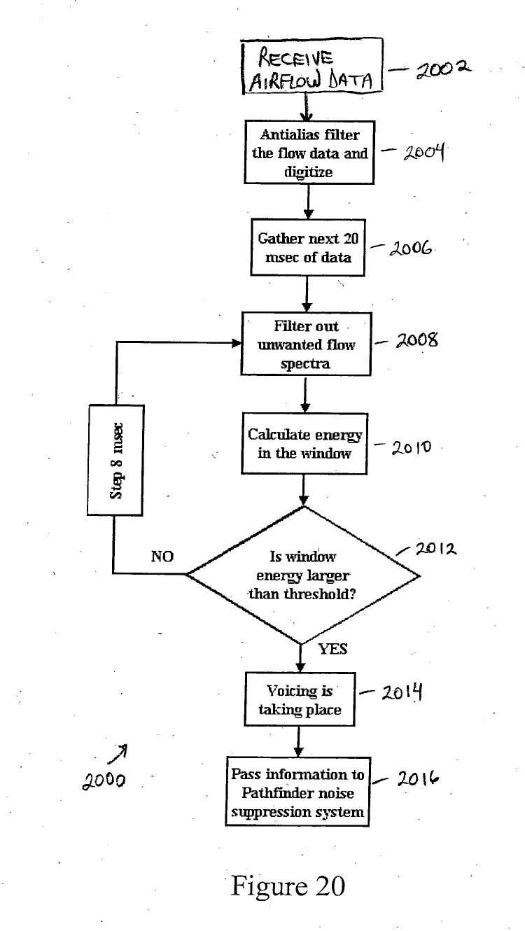

FIG. 20 is a flow diagram 2000 of a method for determining voiced and unvoiced speech using an airflow-based VAD, under an embodiment. Operation begins with the receiving the airflow data, at block 2002. The processing associated with the VAD includes filtering the airflow data to preclude aliasing, and digitizing the filtered data for processing, at block 2004. The digitized data is segmented into windows 20 milliseconds (msec) in length, and the data is stepped 8 msec at a time, at block 2006. The processing further includes filtering the windowed data, at block 2008, to remove low frequency movement and breathing artifacts, as well as other unwanted spectral information. The energy in each window is calculated by summing the squares of the amplitudes as described above, at block 2010.

The calculated energy values are compared to a threshold value, at block 2012. The speech of a window corresponding to the airflow data is designated as voiced speech when the energy of the window is at or above the threshold value, at block 2014. Information of the voiced data is passed to the Pathfinder system for use as VAD information, at block 2016. Noise suppression systems of alternative embodiments can use multiple threshold values to indicate the relative strength or confidence of the voicing signal, but are not so limited.

Manual VAD Devices/Methods

The manual VAD devices of an embodiment include VAD devices that provide the capability for manual activation by a user or observer, for example, using a pushbutton or switch device. Activation of the manual VAD device, or manually overriding an automatic VAD device like those described above, results in generation of a VAD signal.

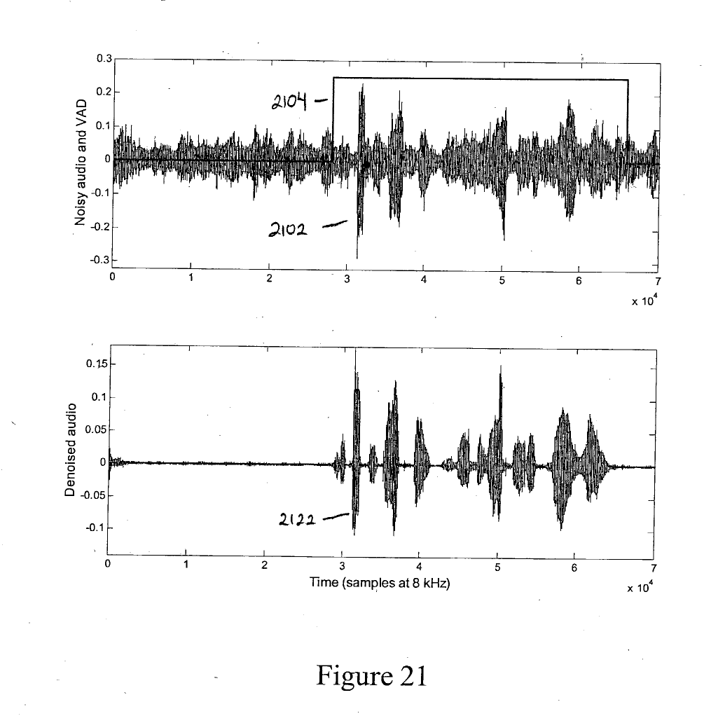

FIG. 21 shows plots including a noisy audio signal 2102 along with a corresponding manually activated/calculated VAD signal 2104, and the denoised audio signal 2122 following processing by the Pathfinder system using the manual VAD signal 2104, under an embodiment. The audio signal 2102 was recorded using an Aliph microphone set in a babble noise environment inside a chamber measuring six (6) feet on a side and having a ceiling height of eight (8) feet. The Pathfinder system is implemented in real-time, with a delay of approximately 10 msec. The difference in the raw audio signal 2102 and the denoised audio signal 2122 clearly show noise suppression approximately in the range of 25-30 dB with little distortion of the desired speech signal. Thus, denoising using the manual VAD information is effective.

Those skilled in the art recognize that numerous electronic systems that process signals including both desired acoustic information and noise can benefit from the VAD devices/methods described above. As an example, an earpiece or headset that includes one of the VAD devices described above can be linked via a wired and/or wireless coupling to a handset like a cellular telephone. Specifically, for example, the earpiece or headset includes the Skin Surface Microphone (SSM) VAD described above to support the Pathfinder system denoising.

As another example, a conventional microphone couples to the handset, where the handset hosts one or more programs that perform VAD determination and denoising. For example, a handset using one or more conventional microphones uses the PVAD and the Pathfinder systems in some combination to perform VAD determination and denoising.

Pathfinder Noise Suppression System

As described above, FIG. 1 is a block diagram of a signal processing system 100 including the Pathfinder noise suppression system 101 and a VAD system 102, under an embodiment. The signal processing system 100 includes two microphones MIC 1 110 and MIC 2 112 that receive signals or information from at least one speech source 120 and at least one noise source 122. The path s(n) from the speech source 120 to MIC 1 and the path n(n) from the noise source 122 to MIC 2 are considered to be unity. Further, H₁(z) represents the path from the noise source 122 to MIC 1, and H₂(z) represents the path from the signal source 120 to MIC 2.

A VAD signal 104, derived in some manner, is used to control the method of noise removal. The acoustic information coming into MIC 1 is denoted by m₁(n). The information coming into MIC 2 is similarly labeled m₂(n). In the z (digital frequency) domain, we can represent them as M₁(z) and M₂(z). Thus

M₁(z)=S(z)+N(z)H₁(z)

M₂(z)=N(z)+S(z)H₂(z) (1)

This is the general case for all realistic two-microphone systems. There is always some leakage of noise into MIC 1, and some leakage of signal into MIC 2. Equation 1 has four unknowns and only two relationships and, therefore, cannot be solved explicitly.

However, perhaps there is some way to solve for some of the unknowns in Equation 1 by other means. Examine the case where the signal is not being generated, that is, where the VAD indicates voicing is not occurring. In this case, s(n)=S(z)=0, and Equation 1 reduces to

M₁ₙ(z)=N(z)H₁(z)

M₂ₙ(z)=N(z)

where the n subscript on the M variables indicate that only noise is being received. This leads to

$$\begin{matrix}{M}_{1 n} ( z ) = {M}_{2 n} ( z ) {H}_{1} ( z ) \\ {H}_{1} ( z ) = \frac{{M}_{1 n} ( z )}{{M}_{2 n} ( z )}\end{matrix} .$$

Now, H₁(z) can be calculated using any of the available system identification algorithms and the microphone outputs when only noise is being received. The calculation should be done adaptively in order to allow the system to track any changes in the noise.

After solving for one of the unknowns in Equation 1, H₂(z) can be solved for by using the VAD to determine when voicing is occurring with little noise. When the VAD indicates voicing, but the recent (on the order of 1 second or so) history of the microphones indicate low levels of noise, assume that n(s)=N(z)˜0. Then Equation 1 reduces to

M₁ₛ(z)=S(z)

M₂ₛ(z)=S(z)H₂(z)

which in turn leads to

$$\begin{matrix}{M}_{2 s} ( z ) = {M}_{1 s} ( z ) {H}_{2} ( z ) \\ {H}_{2} ( z ) = \frac{{M}_{2 s} ( z )}{{M}_{1 s} ( z )}\end{matrix}$$

This calculation for H₂(z) appears to be just the inverse of the H₁(z) calculation, but remember that different inputs are being used. Note that H₂(z) should be relatively constant, as there is always just a single source (the user) and the relative position between the user and the microphones should be relatively constant. Use of a small adaptive gain for the H₂(z) calculation works well and makes the calculation more robust in the presence of noise.

Following the calculation of H₁(z) and H₂(z) above, they are used to remove the noise from the signal. Rewriting Equation 1 as

S(z)=M₁(z)−N(z)H₁(z)

N(z)=M₂(z)−S(z)H₂(z)

S(z)=M₁(z)−[M₂(z)−S(z)H₂(z)]H₁(z)

S(z)]1−H₂(z)H₁(z)]=M₁(z)−M₂(z)H₁(z)

allows solving for S(z)

$$S ( z ) = \frac{{M}_{1} ( z ) - {M}_{2} ( z ) {H}_{1} ( z )}{1 - {H}_{2} ( z ) {H}_{1} ( z )} . \qquad ( 2 )$$

Generally, H₂(z) is quite small, and H₁(z) is less than unity, so for most situations at most frequencies

H₂(z)H₁(z)>>1,

and the signal can be calculated using

S(z)≈M₁(z)−M₂(z)H₁(z) (3)

Therefore the assumption is made that H₂(z) is not needed, and H₁(z) is the only transfer to be calculated. While H₂(z) can be calculated if desired, good microphone placement and orientation can obviate the need for H₂(z) calculation.

Significant noise suppression can only be achieved through the use of multiple subbands in the processing of acoustic signals. This is because most adaptive filters used to calculate transfer functions are of the FIR type, which use only zeros and not poles to calculate a system that contains both zeros and poles as

$${H}_{1} ( z ) {⟶}_{MODELS} \frac{B ( z )}{A ( z )} .$$

Such a model can be sufficiently accurate given enough taps, but this can greatly increase computational cost and convergence time. What generally occurs in an energy-based adaptive filter system such as the least-mean squares (LMS) system is that the system matches the magnitude and phase well at a small range of frequencies that contain more energy than other frequencies. This allows the LMS to fulfill its requirement to minimize the energy of the error to the best of its ability, but this fit may cause the noise in areas outside of the matching frequencies to rise, reducing the effectiveness of the noise suppression.

The use of subbands alleviates this problem. The signals from both the primary and secondary microphones are filtered into multiple subbands, and the resulting data from each subband (which can be frequency shifted and decimated if desired, but it is not necessary) is sent to its own adaptive filter. This forces the adaptive filter to try to fit the data in its own subband, rather than just where the energy is highest in the signal. The noise-suppressed results from each subband can be added together to form the final denoised signal at the end. Keeping everything time-aligned and compensating for filter shifts is not easy, but the result is a much better model to the system at the cost of increased memory and processing requirements.

At first glance, it may seem as if the Pathfinder algorithm is very similar to other algorithms such as classical ANC (adaptive noise cancellation), shown in FIG. 2. However, close examination reveals several areas that make all the difference in terms of noise suppression performance, including using VAD information to control adaptation of the noise suppression system to the received signals, using numerous subbands to ensure adequate convergence across the spectrum of interest, and supporting operation with acoustic signal of interest in the reference microphone of the system, as described in turn below.

Regarding the use of VAD to control adaptation of the noise suppression system to the received signals, classical ANC uses no VAD information. Since, during speech production, there is signal in the reference microphone, adapting the coefficients of H₁(z) (the path from the noise to the primary microphone) during the time of speech production would result in the removal of a large part of the speech energy from the signal of interest. The result is signal distortion and reduction (de-signaling). Therefore, the various methods described above use VAD information to construct a sufficiently accurate VAD to instruct the Pathfinder system when to adapt the coefficients of H₁ (noise only) and H₂ (if needed, when speech is being produced).

An important difference between classical ANC and the Pathfinder system involves subbanding of the acoustic data, as described above. Many subbands are used by the Pathfinder system to support application of the LMS algorithm on information of the subbands individually, thereby ensuring adequate convergence across the spectrum of interest and allowing the Pathfinder system to be effective across the spectrum.

Because the ANC algorithm generally uses the LMS adaptive filter to model H₁, and this model uses all zeros to build filters, it was unlikely that a “real” functioning system could be modeled accurately in this way. Functioning systems almost invariably have both poles and zeros, and therefore have very different frequency responses than those of the LMS filter. Often, the best the LMS can do is to match the phase and magnitude of the real system at a single frequency (or a very small range), so that outside this frequency the model fit is very poor and can result in an increase of noise energy in these areas. Therefore, application of the LMS algorithm across the entire spectrum of the acoustic data of interest often results in degradation of the signal of interest at frequencies with a poor magnitude/phase match.

Finally, the Pathfinder algorithm supports operation with the acoustic signal of interest in the reference microphone of the system. Allowing the acoustic signal to be received by the reference microphone means that the microphones can be much more closely positioned relative to each other (on the order of a centimeter) than in classical ANC configurations. This closer spacing simplifies the adaptive filter calculations and enables more compact microphone configurations/solutions. Also, special microphone configurations have been developed that minimize signal distortion and de-signaling, and support modeling of the signal path between the signal source of interest and the reference microphone.

In an embodiment, the use of directional microphones ensures that the transfer function does not approach unity. Even with directional microphones, some signal is received into the noise microphone. If this is ignored and it is assumed that H₂(z)=0, then, assuming a perfect VAD, there will be some distortion. This can be seen by referring to Equation 2 and solving for the result when H₂(z) is not included:

S(z)[1−H₂(z)H₁(z)]=M₁(z)−M₂(z)H₁(z). (4)

This shows that the signal will be distorted by the factor [1−H₂(z)H₁(z)]. Therefore, the type and amount of distortion will change depending on the noise environment. With very little noise, H₁(z) is approximately zero and there is very little distortion. With noise present, the amount of distortion may change with the type, location, and intensity of the noise source(s). Good microphone configuration design minimizes these distortions.

The calculation of H₁ in each subband is implemented when the VAD indicates that voicing is not occurring or when voicing is occurring but the SNR of the subband is sufficiently low. Conversely, H₂ can be calculated in each subband when the VAD indicates that speech is occurring and the subband SNR is sufficiently high. However, with proper microphone placement and processing, signal distortion can be minimized and only H₁ need be calculated. This significantly reduces the processing required and simplifies the implementation of the Pathfinder algorithm. Where classical ANC does not allow any signal into MIC 2, the Pathfinder algorithm tolerates signal in MIC 2 when using the appropriate microphone configuration. An embodiment of an appropriate microphone configuration, as described above with reference to FIG. 11, is one in which two cardioid unidirectional microphones are used, MIC 1 and MIC 2. The configuration orients MIC 1 toward the user's mouth. Further, the configuration places MIC 2 as close to MIC 1 as possible and orients MIC 2 at 90 degrees with respect to MIC 1.

Perhaps the best way to demonstrate the dependence of the noise suppression on the VAD is to examine the effect of VAD errors on the denoising in the context of a VAD failure. There are two types of errors that can occur. False positives (FP) are when the VAD indicates that voicing has occurred when it has not, and false negatives (FN) are when the VAD does not detect that speech has occurred. False positives are only troublesome if they happen too often, as an occasional FP will only cause the H₁ coefficients to stop updating briefly, and experience has shown that this does not appreciably affect the noise suppression performance. False negatives, on the other hand, can cause problems, especially if the SNR of the missed speech is high.

Assuming that there is speech and noise in both microphones of the system, and the system only detects the noise because the VAD failed and returned a false negative, the signal at MIC 2 is

M₂=H₁N+H₂S,

where the z's have been suppressed for clarity. Since the VAD indicates only the presence of noise, the system attempts to model the system above as a single noise and a single transfer function according to

TFmodel=\tilde{H}₁Ñ.

The Pathfinder system uses an LMS algorithm to calculate \tilde{H}₁, but the LMS algorithm is generally best at modeling time-invariant, all-zero systems. Since it is unlikely that the noise and speech signal are correlated, the system generally models either the speech and its associated transfer function or the noise and its associated transfer function, depending on the SNR of the data in MIC 1, the ability to model H₁ and H₂, and the time-invariance of H₁ and H₂, as described below.

Regarding the SNR of the data in MIC 1, a very low SNR (less than zero (0)) tends to cause the Pathfinder system to converge to the noise transfer function. In contrast, a high SNR (greater than zero (0)) tends to cause the Pathfinder system converge to the speech transfer function. As for the ability to model H₁, if either H₁ or H₂ is more easily modeled using LMS (an all-zero model), the Pathfinder system tends to converge to that respective transfer function.

In describing the dependence of the system modeling on the time-invariance of H₁ and H₂, consider that LMS is best at modeling time-invariant systems. Thus, the Pathfinder system would generally tend to converge to H₂, since H₂ changes much more slowly than H₁ is likely to change.

If the LMS models the speech transfer function over the noise transfer function, then the speech is classified as noise and removed as long as the coefficients of the LMS filter remain the same or are similar. Therefore, after the Pathfinder system has converged to a model of the speech transfer function H₂ (which can occur on the order of a few milliseconds), any subsequent speech (even speech where the VAD has not failed) has energy removed from it as well as the system “assumes” that this speech is noise because its transfer function is similar to the one modeled when the VAD failed. In this case, where H₂ is primarily being modeled, the noise will either be unaffected or only partially removed.

The end result of the process is a reduction in volume and distortion of the cleaned speech, the severity of which is determined by the variables described above. If the system tends to converge to H₁, the subsequent gain loss and distortion of the speech will not be significant. If, however, the system tends to converge to H₂, then the speech can be severely distorted.

This VAD failure analysis does not attempt to describe the subtleties associated with the use of subbands and the location, type, and orientation of the microphones, but is meant to convey the importance of the VAD to the denoising. The results above are applicable to a single subband or an arbitrary number of subbands, because the interactions in each subband are the same.

In addition, the dependence on the VAD and the problems arising from VAD errors described in the above VAD failure analysis are not limited to the Pathfinder noise suppression system. Any adaptive filter noise suppression system that uses a VAD to determine how to denoise will be similarly affected. In this disclosure, when the Pathfinder noise suppression system is referred to, it should be kept in mind that all noise suppression systems that use multiple microphones to estimate the noise waveform and subtract it from a signal including both speech and noise, and that depend on VAD for reliable operation, are included in that reference. Pathfinder is simply a convenient referenced implementation.

The VAD devices and methods described above for use with noise suppression systems like the Pathfinder system include a system for denoising acoustic signals, wherein the system comprises: a denoising subsystem including at least one receiver coupled to provide acoustic signals of an environment to components of the denoising subsystem; a voice detection subsystem coupled to the denoising subsystem, the voice detection subsystem receiving voice activity signals that include information of human voicing activity, wherein components of the voice detection subsystem automatically generate control signals using information of the voice activity signals, wherein components of the denoising subsystem automatically select at least one denoising method appropriate to data of at least one frequency subband of the acoustic signals using the control signals, and wherein components of the denoising subsystem process the acoustic signals using the selected denoising method to generate denoised acoustic signals.

The receiver of an embodiment of the denoising subsystem couples to at least one microphone array that detects the acoustic signals.

The microphone array of an embodiment includes at least two closely-spaced microphones.

The voice detection subsystem of an embodiment receives the voice activity signals via a sensor, wherein the sensor is selected from among at least one of an accelerometer, a skin surface microphone in physical contact with skin of a user, a human tissue vibration detector, a radio frequency (RF) vibration detector, a laser vibration detector, an electroglottograph (EGG) device, and a computer vision tissue vibration detector.

The voice detection subsystem of an embodiment receives the voice activity signals via a microphone array coupled to the receiver, the microphone array including at least one of a microphone, a gradient microphone, and a pair of unidirectional microphones.

The voice detection subsystem of an embodiment receives the voice activity signals via a microphone array coupled to the receiver, wherein the microphone array includes a first unidirectional microphone co-located with a second unidirectional microphone, wherein the first unidirectional microphone is oriented so that a spatial response curve maximum of the first unidirectional microphone is approximately in a range of 45 to 180 degrees in azimuth from a spatial response curve maximum of the second unidirectional microphone.

The voice detection subsystem of an embodiment receives the voice activity signals via a microphone array coupled to the receiver, wherein the microphone array includes a first unidirectional microphone positioned colinearly with a second unidirectional microphone.

The VAD methods described above for use with noise suppression systems like the Pathfinder system include a method for denoising acoustic signals, wherein the method comprises: receiving acoustic signals and voice activity signals; automatically generating control signals from data of the voice activity signals; automatically selecting at least one denoising method appropriate to data of at least one frequency subband of the acoustic signals using the control signals; and applying the selected denoising method and generating the denoised acoustic signals.

In an embodiment, selecting further comprises selecting a first denoising method for frequency subbands that include voiced speech.

In an embodiment, selecting further comprises selecting a second denoising method for frequency subbands that include unvoiced speech.

In an embodiment, selecting further comprises selecting a denoising method for frequency subbands devoid of speech.

In an embodiment, selecting further comprises selecting a denoising method in response to noise information of the received acoustic signal, wherein the noise information includes at least one of noise amplitude, noise type, and noise orientation relative to a speaker.

In an embodiment, selecting further comprises selecting a denoising method in response to noise information of the received acoustic signal, wherein the noise information includes noise source motion relative to a speaker.

The VAD methods described above for use with noise suppression systems like the Pathfinder system include a method for removing noise from acoustic signals, wherein the method comprises: receiving acoustic signals; receiving information associated with human voicing activity; generating at least one control signal for use in controlling removal of noise from the acoustic signals; in response to the control signal, automatically generating at least one transfer function for use in processing the acoustic signals in at least one frequency subband; applying the generated transfer function to the acoustic signals; and removing noise from the acoustic signals.

The method of an embodiment further comprises dividing the received acoustic signals into a plurality of frequency subbands.

In an embodiment, generating the transfer function further comprises adapting coefficients of at least one first transfer function representative of the acoustic signals of a subband when the control signal indicates that voicing information is absent from the acoustic signals of a subband.

In an embodiment, generating the transfer funcation further comprises generating at least one second transfer function representative of the acoustic signals of a subband when the control signal indicates that voicing information is present in the acoustic signals of a subband.

In an embodiment, applying the generated transfer function further comprises generating a noise waveform estimate associated with noise of the acoustic signals, and subtracting the noise waveform estimate from the acoustic signal when the acoustic signal includes speech and noise.

Aspects of the invention may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the invention include: microcontrollers with memory (such as electronically erasable programmable read only memory (EEPROM)), embedded microprocessors, firmware, software, etc. If aspects of the invention are embodied as software at least one stage during manufacturing (e.g. before being embedded in firmware or in a PLD), the software may be carried by any computer readable medium, such as magnetically- or optically-readable disks (fixed or floppy), modulated on a carrier signal or otherwise transmitted, etc.

Furthermore, aspects of the invention may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, shall refer to this application as a whole and not to any particular portions of this application. When the word “or” is used in reference to a list of two or more items, that word covers all of the following interpretations of the word: any of the items in the list, all of the items in the list and any combination of the items in the list.

The above descriptions of embodiments of the invention are not intended to be exhaustive or to limit the invention to the precise forms disclosed. While specific embodiments of, and examples for, the invention are described herein for illustrative purposes, various equivalent modifications are possible within the scope of the invention, as those skilled in the relevant art will recognize. The teachings of the invention provided herein can be applied to other processing systems and communication systems, not only for the processing systems described above.

The elements and acts of the various embodiments described above can be combined to provide further embodiments. These and other changes can be made to the invention in light of the above detailed description.

All of the above references and United States patent applications are incorporated herein by reference. Aspects of the invention can be modified, if necessary, to employ the systems, functions and concepts of the various patents and applications described above to provide yet further embodiments of the invention.

In general, in the following claims, the terms used should not be construed to limit the invention to the specific embodiments disclosed in the specification and the claims, but should be construed to include all processing systems that operate under the claims to provide a method for compressing and decompressing data files or streams. Accordingly, the invention is not limited by the disclosure, but instead the scope of the invention is to be determined entirely by the claims.

While certain aspects of the invention are presented below in certain claim forms, the inventors contemplate the various aspects of the invention in any number of claim forms. For example, while only one aspect of the invention is recited as embodied in a computer-readable medium, other aspects may likewise be embodied in a computer-readable medium. Accordingly, the inventors reserve the right to add additional claims after filing the application to pursue such additional claim forms for other aspects of the invention.

What we claim is:

1. A system for denoising acoustic signals, comprising:
a denoising subsystem including at least one receiver coupled to provide acoustic signals of an environment to components of the denoising subsystem;
a voice detection subsystem coupled to the denoising subsystem, the voice detection subsystem receiving voice activity signals that include information of human voicing activity, wherein components of the voice detection subsystem automatically generate control signals using information of the voice activity signals,
wherein components of the denoising subsystem automatically select at least one denoising method appropriate to data of at least one frequency subband of the acoustic signals using the control signals; and
wherein components of the denoising subsystem process the acoustic signals using the selected denoising method to generate denoised acoustic signals.

2. The system of claim 1, wherein the receiver couples to at least one microphone array that detects the acoustic signals.

3. The system of claim 2, wherein the microphone array includes at least two closely-spaced microphones.

4. The system of claim 1, wherein the voice detection subsystem receives the voice activity signals via a sensor, wherein the sensor is selected from among at least one of an accelerometer, a skin surface microphone in physical contact with skin of a user, a human tissue vibration detector, a radio frequency (RF) vibration detector, a laser vibration detector, an electroglottograph (EGG) device, and a computer vision tissue vibration detector.

5. The system of claim 1, wherein the voice detection subsystem receives the voice activity signals via a microphone array coupled to the receiver, the microphone array including at least one of a microphone, a gradient microphone, and a pair of unidirectional microphones.

6. The system of claim 1, wherein the voice detection subsystem receives the voice activity signals via a microphone array coupled to the receiver, wherein the microphone array includes a first unidirectional microphone co-located with a second unidirectional microphone, wherein the first unidirectional microphone is oriented so that a spatial response curve maximum of the first unidirectional microphone is approximately in a range of 45 to 180 degrees in azimuth from a spatial response curve maximum of the second unidirectional microphone.

7. The system of claim 1, wherein the voice detection subsystem receives the voice activity signals via a microphone array coupled to the receiver, wherein the microphone array includes a first unidirectional microphone positioned colinearly with a second unidirectional microphone.

8. A method for denoising acoustic signals, comprising:
receiving acoustic signals and voice activity signals;
automatically generating control signals from data of the voice activity signals;
automatically selecting at least one denoising method appropriate to data of at least one frequency subband of the acoustic signals using the control signals; and
applying the selected denoising method and generating the denoised acoustic signals.

9. The method of claim 8, wherein selecting further comprises selecting a first denoising method for frequency subbands that include voiced speech.

10. The method of claim 9, wherein selecting further comprises selecting a second denoising method for frequency subbands that include unvoiced speech.

11. The method of claim 8, wherein selecting further comprises selecting a denoising method for frequency subbands devoid of speech.

12. The method of claim 8, wherein selecting further comprises selecting a denoising method in response to noise information of the received acoustic signal, wherein the noise information includes at least one of noise amplitude, noise type, and noise orientation relative to a speaker.

13. The method of claim 8, wherein selecting further comprises selecting a denoising method in response to noise information of the received acoustic signal, wherein the noise information includes noise source motion relative to a speaker.

14. A method for removing noise from acoustic signals, comprising:
receiving acoustic signals;
receiving information associated with human voicing activity;
generating at least one control signal for use in controlling removal of noise from the acoustic signals;
in response to the control signal, automatically generating at least one transfer function for use in processing the acoustic signals in at least one frequency subband;
applying the generated transfer function to the acoustic signals; and
removing noise from the acoustic signals.

15. The method of claim 14, further comprising dividing the received acoustic signals into a plurality of frequency subbands.

16. The method of claim 14, wherein generating the transfer function further comprises adapting coefficients of at least one first transfer function representative of the acoustic signals of a subband when the control signal indicates that voicing information is absent from the acoustic signals of a subband.

17. The method of claim 14, wherein generating the transfer funcation further comprises generating at least one second transfer function representative of the acoustic signals of a subband when the control signal indicates that voicing information is present in the acoustic signals of a subband.

18. The method of claim 14, wherein applying the generated transfer function further comprises:
generating a noise waveform estimate associated with noise of the acoustic signals; and
subtracting the noise waveform estimate from the acoustic signal when the acoustic signal includes speech and noise.
